(19) United States
(12) Patent Application Publication
(10) Pub. No.: US 2024/0117449 A1
(43) Pub. Date: Apr. 11, 2024

(54) Detecting Listeria Monocytogenes with Helicase-Dependent Amplification Assay

(71) Applicant: Neogen Corporation, Lansing, MI (US)

(72) Inventors: Katie Adolphsen, Lansing, MI (US); Wesley Colangelo, Brighton, MI (US); Ariel Fox, Lansing, MI (US); Lauren Sangster, Flint, MI (US); Jolien Weytens, New Lothrop, MI (US)

(21) Appl. No.: 18/479,259

(22) Filed: Oct. 2, 2023

Related U.S. Application Data
(60) Provisional application No. 63/378,037, filed on Sep. 30, 2022.

(51) Int. Cl. C12Q 1/689

(52) U.S. Cl. CPC C12Q 1/689

(57) ABSTRACT

The present disclosure provides compositions, methods and kits for detection of L. monocytogenes from a sample, such as an environmental sample.

### CROSS-REFERENCE TO RELATED APPLICATIONS

This U.S. patent application claims priority to U.S. Provisional Application 63/378,037 filed on Sep. 30, 2022, the disclosure of which is considered part of the disclosure of this application and is hereby incorporated by reference in its entirety.

### REFERENCE TO THE SEQUENCE LISTING

The instant application contains a Sequence Listing which has been submitted electronically herewith in ST.26 format and which is hereby incorporated by reference in its entirety. Said ST.26 file was created on Sep. 30, 2023, is named 234994-535873 Sequence-listing and is 72,419 bytes in size.

### TECHNICAL FIELD

The present disclosure relates to detection of Listeria monocytogenes.

### BACKGROUND

Bacterial contamination and infection can pose a serious problem for public health. For example, Listeria monocytogenes (L. monocytogenes) is a harmful pathogen that can be especially problematic in the food industry. There is a need for methods and tools to rapidly detect Listeria monocytogenes in test samples, including for food safety testing and environmental monitoring.

### SUMMARY OF THE INVENTION

The present disclosure provides, in part, compositions, methods, and kits for rapidly detecting Listeria monocytogenes in samples, such as environmental samples. In various aspects, the provided compositions, methods, and kits enable determining presence or absence of Listeria monocytogenes in environmental samples without enrichment or prolonged incubation prior to performing the detection assay itself. Among other things, a benefit of the provided methods is that results can be obtained in a short time (e.g., about 1-2 hours from sample collection), compared to conventional methods that may take about 16-32 hours or more. Due to low sensitivity, conventional methods require enrichment of the sample to increase the concentration of target organisms, or other types of prolonged incubation steps required to increase the concentration of target molecules to detectable levels. The present disclosure provides improvements and benefits compared to such conventional methods.

### BRIEF DESCRIPTION OF THE DRAWINGS

These and other features, aspects, and advantages of the present disclosure may be better understood when the following detailed description is read with reference to the accompanying drawings.

FIG. 1A-FIG. 1B show schematics of a sequence alignment, oligonucleotide locations and amplicon sequence.

FIG. 1A RNA sequences for bacterium of interest (Listeria monocytogenes) and other bacteria were obtained from NCBI and aligned using Geneious Prime software. Shown are the locations of the primers. Oligonucleotides of the present disclosure were designed against Listeria monocytogenes 16S rRNA sequence (e.g., as shown in Table 1A). The SEQ ID NOs for the full 16S bacterial sequences, primers, and probe are presented in FIG. 10.

FIG. 1B L. monocytogenes amplicon sequence. Blocked forward primer eF2 (SEQ ID NO: 1), the blocked reverse, R3 (SEQ ID NO: 2), unblocked reverse R12 (SEQ ID NO: 3), test probe Probe? (SEQ ID NO: 5) and the TseI restriction site is diagramed. Two possible amplicons can be produced by the introduction of two reverse primers. Amplicons made with the unblocked reverse primer will have 108 bp and amplicons synthesized from the blocked reverse primer will have 94 bp (SEQ ID NO: 4).

FIG. 2 is a graph showing amplification signals observed with blocked forward and blocked reverse primers with DNA and RNA templates. L. monocytogenes gene block fragment has robust signal with two blocked primers. 1 ng of purified L. monocytogenes RNA tested with two blocked primers resulted in no amplification indicating reverse transcriptase requires a standard reverse primer. An additional HDA template and primers were tested as a positive control for the HDA components. The no template control had very delayed signal, indicative of primer dimers. See figure legend for curve identification.

FIG. 3A-FIG. 3B are graphs showing amplification of target L. monocytogenes nucleic acids from an RNA template using a combination of blocked and unblocked reverse primers.

FIG. 3A shows that 64 nM of R3 and 16 nM of R12 allowed for strong L. monocytogenes signal with minimal signal from L. innocua. The no template control (NTC) results are characteristic of primer dimer formation. See figure legend for curve identification.

FIG. 3B is a melt curve also displays the desired product in the L. monocytogenes and no defined peak in the L. innocua indicating the L. innocua target is not being amplified. See figure legend for curve identification.

FIG. 4 is a graph showing detection of amplified L. monocytogenes nucleic acids using a selective detection probe in an HDA assay with purified RNA starting material.

FIG. 5 is a graph showing results from a limit of detection assay with serially diluted purified L. monocytogenes RNA.

FIG. 6 is a graph showing results from inclusivity testing for two serotypes of L. monocytogenes (serotypes 1/2a and 4c). An RNA positive control and a no template negative control were included.

FIG. 7A-FIG. 7B are graphs showing amplification of target L. monocytogenes nucleic acids using a detection assay with a mechanical lysis procedure. Lyophilized L. monocytogenes reactions were used to determine the appropriate size and mass of Zirconia Silicate beads.

FIG. 7A The 0.5 mm Zirconia Silicate beads both resulted in positive at 25 mg, 50 mg, 75 mg, 100 mg.

FIG. 7B The 0.1 mm Zirconia Silicate beads both resulted in positive at 25 mg, 50 mg, 75 mg, 100 mg.

FIG. 8 presents graphs showing amplification and limit of detection testing for target L. monocytogenes nucleic acids using a detection assay with mechanical bead lysis. Disruption time was tested at 2 and 5 minutes.

FIG. 9A-FIG. 9B are graphs showing an L. monocytogenes detection assay with a semi-competitive control template.

FIG. 9A shows amplification detected with the ROX channel. The test reaction was positive for serially diluted purified L. monocytogenes RNA down to 100 fg. 1 ng of L. innocua purified RNA resulted in a negative test signal while maintaining a strong control signal.

FIG. 9B shows amplification detected with the HEX (control) channel. The internal control reaction is sufficient for all samples.

FIG. 10 shows alignment of 16S rRNA sequences, a consensus sequence used in the present disclosure, and sequences of primers and probe.

### DETAILED DESCRIPTION

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference and understanding, and the inclusion of such definitions herein should not necessarily be construed to mean a substantial difference over what is generally understood in the art. Articles such as “a,” “an,” and “the” may mean one or more than one unless indicated to the contrary or otherwise evident from the context. The term “and/or” means one or all of the listed elements or a combination of any two or more of the listed elements. As appropriate, procedures involving the use of commercially available kits and/or reagents are generally carried out in accordance with manufacturer's guidance and/or protocols and/or parameters unless otherwise noted. The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

About: The word “about” is understood to modify numbers recited in the specification and claims whether or not explicitly stated. The term “about” is intended to take into account standard measurement errors and encompass rounding off

Aliquot: An “aliquot” as used with reference to an aliquot of a sample or lysate means a portion or all of the stated sample or lysate that is used for further processing, such as downstream steps in a method.

Control RNA: As used herein, the term “control RNA” refers to an RNA molecule added to a detection mixture or an assay mixture for purposes of serving as a positive internal control for amplification and/or detection by methods described herein. A control RNA can be detected with a control probe. As used herein, a “control probe” is a probe that is designed to detect amplification products of the control RNA.

Downstream methods: As used herein, the phrase “downstream methods” or “downstream assays” refers to one or more additional methods (e.g., one or more additional steps) or assays (e.g., a detection assay) that are carried out. The one or more additional methods or assays may be carried out after the stated procedure is initiated, or after the stated procedure has been performed. For example, a method that is downstream to cell lysis may be carried out after lysis begins, and/or after a lysis step generates a lysate. A downstream method or downstream assay does not necessarily follow directly after the stated procedure. For example, additional intervening steps may be included between the stated procedure and the downstream method or assay.

Enrichment: The term “enrichment” refers to expansion of target cells (e.g., target bacteria cells). Enrichment may comprise exposing a sample comprising target bacteria cells to cell growth conditions that promote increasing the number of the target bacteria cells. Some methods of enrichment comprise use of a broth, which may be referred to as an “enrichment broth.” An enrichment broth may comprise both beneficial compounds for the growth of the target microbe (e.g., target bacteria), as well as inhibitory compounds for other microbes that are detrimental to the growth of the target microbe or to downstream assay steps. Continuing enrichment of the target microbe on different selective media is a known method of assaying for the presence of the target microbe. Methods known in the art, for example for food samples or food production environments or pharmaceutical environments, can be found in the USDA Microbiology Laboratory Guidebook (USDA-MLG), or the U.S. Food and Drug Administration (FDA) Bacteriological Analytical Manual (FDA-BAM).

Environmental sample: As used herein, the term “environmental sample” refers to a sample collected from the environment. In preferred embodiments, an environmental sample is collected from the environment in a room or a building where foods and/or beverages are produced and/or processed, such as that of a manufacturing plant or a commercial kitchen. In some preferred embodiments, environmental samples may be collected from food/beverage production, processing and/or service sites. Environmental samples may be collected from both food contact surfaces (e.g., slicers, mixers, utensils or conveyors) and non-food contact surfaces (e.g., floors, drains, carts or equipment housing). Environmental surfaces may be composed of a variety of materials or combinations of materials, such as stainless steel, plastic, ceramic tile, sealed concrete, or rubber. In some embodiments, environmental samples may be collected from industrial food equipment surfaces. Additional examples of environmental samples are provided below.

Helicase: The term “helicase” refers herein to an enzyme capable of unwinding a double-stranded nucleic acid enzymatically. For example, helicases are enzymes that are found in all organisms and in all processes that involve nucleic acids such as replication, recombination, repair, transcription, translation and RNA splicing. Helicases use the energy of nucleoside triphosphate (for example ATP) hydrolysis to break the hydrogen bonds that hold the strands together in duplex DNA and RNA. A helicase may translocate along DNA or RNA in a 5′ to 3′ direction or in the opposite 3′ to 5′ direction. Helicases can be found in prokaryotes, viruses, archaea, and eukaryotes or recombinant forms of naturally occurring enzymes as well as analogues or derivatives having the specified activity. Examples of naturally occurring DNA helicases, described by Kornberg and Baker in chapter 11 of their book, DNA Replication, W.H. Freeman and Company (2^{nd} ed. (1992)), include E. coli helicase I, II, III, & IV, Rep, DnaB, PriA, PcrA, T4 Gp41 helicase, T4 Dda helicase, T7 Gp4 helicases, SV40 Large T antigen, yeast RAD. Additional helicases include RecQ helicase (Harmon and Kowalczykowski, J. Biol. Chem. 276:232-243 (2001)), thermostable UvrD helicases from T. tengcongensis and T. thermophilus (Collins and McCarthy, Extremophiles. 7:35-41. (2003)), thermostable DnaB helicase from T. aquaticus (Kaplan and Steitz, J. Biol. Chem. 274:6889-6897 (1999)), and MCM helicase from archaeal and eukaryotic organisms (Grainge et al., Nucleic Acids Res. 31:4888-4898 (2003)).

Helicase-dependent amplification: The term “helicase-dependent amplification” or “HDA” refers to an in vitro method for amplifying nucleic acids by using a helicase for unwinding a double-stranded nucleic acid to generate templates for primer hybridization and subsequent primer-extension. This process utilizes two oligonucleotide primers, each hybridizing to the 3′-end of either the sense strand containing the target sequence or the anti-sense strand containing the reverse complementary target sequence. The HDA reaction is a general method for helicase-dependent nucleic acid amplification.

Hybridization: The term “hybridization” refers to binding of a single-stranded nucleic acid to a complementary single-stranded nucleic acid, preferably under conditions in which binding occurs only specifically to a nucleic acid region having a complementary sequence and not to other regions. In some embodiments, hybridization occurs between an oligonucleotide and a complementary region of a single-stranded nucleic acid. The specificity of hybridization may be influenced by the length of the oligonucleotide, the temperature in which the hybridization reaction is performed, the ionic strength, and the pH. In some embodiments, hybridization occurs between a primer and a complementary single-stranded region on a target nucleic acid to facilitate polymerase-dependent replication of the target nucleic acid and/or reverse-transcriptase-dependent synthesis of cDNA from a target nucleic acid serving as an RNA template. In some embodiments, hybridization occurs between a probe and a nucleic acid of interest. In some embodiments, hybridization refers to binding of an oligonucleotide primer to a region of the single-stranded nucleic acid template under conditions in which the primer binds only specifically to its complementary sequence on one of the template strands, not other regions in the template. In some embodiments, hybridization occurs between two single-stranded nucleic acids that are at least 95%, at least 96%, at least 97%, at least 98%, at least 99% or 100% complementary.

Hybridization specificity: As used herein, the term “hybridization specificity” refers to the ability of a molecule comprising or consisting of a single-stranded polynucleotide, or a portion of said molecule, to anneal to a complementary region of a polynucleotide. The degree of hybridization may vary depending on conditions (such as temperature, pH, buffers, etc.) and depending on level of complementarity. In some embodiments, a molecule or portion thereof, may have hybridization specificity for a region of a polynucleotide that is at least 95%, at least 96%, at least 97%, at least 98%, at least 99% or 100% complementary to the stated molecule or portion thereof

Identity: The term “identity” as known in the art, refers to a relationship between two or more sequences, as determined by comparing the sequences. In the art, identity also means the degree of sequence relatedness between sequences, as determined by the number of matches between strings of two or more residues (amino acid or nucleic acid). Identity measures the percent of identical matches between two or more sequences with gap alignments (if any) addressed by a particular mathematical model or computer program (i.e., “algorithms”). Identity of related sequences can be readily calculated by known methods. Percent identity may be determined, for example, by comparing sequence information using sequence alignment programs known to those skilled in the art.

Incubation: The term “incubation” refers to the process of exposing something (e.g., a sample) to a set of conditions (e.g., temperature, specific reagents, etc.) for a period of time. As used herein, a “prolonged incubation” refers to incubation for a period of time that is longer than at least one hour or at least two hours.

Isothermal: The term “isothermal” used in the context of helicase-dependent amplification refers to nucleic acid amplification that occurs at a constant temperature.

Lysis: As used herein, “lyse” or “lysis” refers to breaking open cells (e.g., bacterial cells) to release its contents (e.g., nucleic acids). Lysis may be achieved by contacting a sample comprising cells with a lysis buffer comprising lytic components. Examples of lytic components may be, but are not limited to, detergents, enzymes or denaturing salts. Alternatively, lysis may be achieved by a mechanical lysis procedure. An example of a mechanical lysis procedure includes use of beads (e.g., Zirconia Silicate beads) to disrupt cells to release their contents. A fluid comprising the contents of lysed cells is referred to as a “lysate.”

Lytic enzyme: As used herein, the term “lytic enzyme” refers to any enzyme that promotes lysis of a cell. Preferably, the cell is a bacterial cell. For example, lytic enzymes promote lysis of a bacterial cell by hydrolyzing the bacterial cell wall. Non-limiting examples of lytic enzymes are lysozyme and mutanolysin.

Melting: The terms “melting”, “unwinding” or “denaturing” refer to separating all or part of two complementary strands of a nucleic acid duplex.

Nucleic acid: The terms “nucleic acid molecule,” “nucleic acid,” “oligonucleotide,” and “polynucleotide” may be used interchangeably, and refer to a polymer of nucleotides. Such polymers of nucleotides may contain natural and/or non-natural nucleotides. Illustrative nucleic acids or polynucleotides include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs) or hybrids thereof “Nucleic acid sequence” refers to the linear sequence of nucleotides of the nucleic acid molecule or polynucleotide. In some instances, the nucleic acid may be a short molecule (approximately 13-25 nucleotides long and/or less than 200 nucleotide residues) and may then be termed an “oligonucleotide.” The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules, plasmids, supercoiled DNA and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). DNA includes, but is not limited to, cDNA, genomic DNA, plasmid DNA, synthetic DNA, and semi-synthetic DNA. Those molecules which are double stranded nucleic acid molecules may be nicked or intact. The double stranded or single-stranded nucleic acid molecules may be linear or circular. The duplexes may be blunt ended or have single-stranded tails. The term “duplex” refers to a nucleic acid molecule that is double-stranded in whole or part. The single-stranded molecules may have secondary structure in the form of hairpins or loops and stems. Nucleic acids may be isolated, cloned or synthesized in vitro by means of chemical synthesis. Any of the above described nucleic acids may be subject to modification where individual nucleotides within the nucleic acid are chemically altered (for example, by methylation). Modifications may arise naturally or by in vitro synthesis.

Presence or absence: The phrase “presence or absence” when used in reference to detecting presence or absence of amplified nucleic acid or determining presence or absence of a target bacteria, refers to the state of having or not having the stated composition (e.g., amplified nucleic acid or target bacteria). The presence and absence assay signal rely on the limit of detection of the assay. As used herein, “absence” does not necessarily mean absolute absence; instead, a composition may be determined as absent if there is a low level, such as below a negative control threshold, that is detected or if the concentration of the composition in a test sample is close to the limit of detection or much lower than the limit of detection of the assay. As used herein, “presence” may be determined by a specific cut-off, below which the composition is not considered to be present. For samples with concentrations close to the limit of detection, multiple replicates may be necessary to detect presence.

Primer: As used herein, the term “primer” refers to a single-stranded nucleic acid capable of binding to a single-stranded region on a target nucleic acid to facilitate polymerase-dependent replication of the target nucleic acid and/or reverse-transcriptase-dependent synthesis of cDNA from a target nucleic acid serving as an RNA template. In some embodiments, a primer is capable of binding to a region on an RNA molecule. In some embodiments, a primer is capable of binding to a single-stranded region on a DNA or cDNA molecule. In some embodiments, a primer is capable of binding to both a single-stranded region on an RNA molecule and a single-stranded region on a DNA or cDNA molecule. In some embodiments, a primer may comprise one or more modifications. A non-limiting example of a modification is a chemical moiety linked to the nucleic acids of the primer. In some embodiments, a primer is a blocked primer. In some embodiments, a blocked primer comprises a blocking group. Illustrative non-limiting examples of blocking groups are described in U.S. Pat. No. 10,227,641. In some embodiments, a blocking group prevents primer extension and/or inhibits the blocked primer from serving as a template for DNA synthesis. The term “primer pair” refers to a set of two primers, one serving as the forward primer and the other as the reverse primer, each binding to one of the two ends of a single-stranded region on a target nucleic acid. A primer of the present disclosure generally has less than 50 residues. Preferably, a primer of the present disclosure is in a size range having a lower limit of about 5 to about 15 residues and an upper limit of about 25 to about 35 residues. For example, a primer of the present disclosure may comprise 14, 15, 16, 17, 18, 19, 20, 21, 22, 23, 24, 25, 26, 27, 28, or 29 residues.

Probe: As used herein, the term “probe” refers to a labeled molecule or portion thereof that is designed to detect a nucleic acid of interest. In some embodiments, the term “probe” is interchangeable with the term “molecular probe.” When used in reference to a probe, the term “detect” is interchangeable with the term “recognize” and refers to the ability of the probe to identify the nucleic acid of interest. In some embodiments, a probe is a single-stranded nucleic acid comprising one or more complementary sequences to the nucleic acid of interest. When the probe is placed in contact with a sample under conditions that allow the probe to hybridize with the nucleic acid of interest, the nucleic acid of interest is detected. The label of the probe can be a tag, such as a radioactive or chemical tag, that allows hybridization of the probe to the nucleic acid of interest to be visualized. In some embodiments, the probe is a fluorescent molecular probe (also referred to herein as a “fluorescent probe”), which is a probe that emits fluorescence. An example of a fluorescent molecular probe is a conditional fluorescent hybridization probe. In some embodiments, the probe is a conditional fluorescent hybridization probe that emits fluorescence when hybridized to the nucleic acid of interest. In some embodiments, the nucleic acid of interest is an amplicon produced by helicase-dependent amplification (I-IDA) according to methods of the present disclosure.

Sensitivity: The term “sensitivity” when used in reference to a detection method or assay is the proportion of actual positive samples that are correctly identified as positive by the method or assay. Actual positive samples are generally defined by using a validated assay used to detect the presence of a target microbe (e.g., target bacteria). In some embodiments the actual positives are defined by a method comprising culturing a sample to determine whether target microbes are present. In some embodiments, one or more positive control samples are used as actual positive samples. In some embodiments, one or more positive control samples are used alongside test samples that may or may not be actual positive samples. In some embodiments, a method is described as having “sufficient sensitivity,” which refers to the ability of the method to correctly identify as positive an actual positive sample. In some embodiments, a method may have sufficient sensitivity under certain conditions.

Specificity: The term “specificity” when used in reference to a detection method or assay is the proportion of actual negative samples that are correctly identified as negative by the method or assay. Actual negative samples are generally defined by using a validated assay used to detect the presence of a target microbe (e.g., target bacteria). In some embodiments the actual negatives are defined by a method comprising culturing a sample to determine whether target microbes are present. In some embodiments, one or more negative control samples are used as actual negative samples. In some embodiments, one or more negative control samples are used alongside test samples that may or may not be actual negative samples. In some embodiments, a negative control sample comprises a bacterium that is not the target bacterium.

Target bacteria: The term “target bacteria,” as used herein, refers to one or more species of bacteria that are targeted for detection and/or quantification in a sample, such as an environmental sample. In some embodiments, the target bacteria are any bacteria of the species L. monocytogenes.

Target nucleic acid: According to the present disclosure, a “target nucleic acid,” refers to a nucleic acid molecule, or portion thereof, that is present in target bacteria. In some embodiments the target nucleic acid is detected using nucleic acid detection methods. Methods for detecting a target nucleic acid may be used for purposes of determining the presence or absence of the target bacteria in a sample. In some embodiments, a target nucleic acid is detected using the compositions and methods provided by the present disclosure. In some embodiments, a target nucleic acid is detected using methods comprising helicase-dependent amplification (HDA). In such cases, the target nucleic acid is amplified according to HDA methods and is referred to as an HDA target nucleic acid. Specifically, the term “HDA target nucleic acid” refers to a whole or part of nucleic acid to be selectively amplified and which is defined by 3′ and 5′ boundaries. The HDA target nucleic acid may also be referred to as a fragment or sequence that is intended to be amplified. The size of the HDA target nucleic acid to be amplified may be, for example, in the range of about 50 base pairs (bp) to about 5000 bp. In preferred embodiments, the size of the HDA target nucleic acid to be amplified is 50-150 bp.

The HDA target nucleic acid may be contained within a longer double-stranded or single-stranded nucleic acid. Alternatively, the HDA target nucleic acid may be an entire double-stranded or single-stranded nucleic acid. If the initial nucleic acid provided for an HDA method is RNA, the RNA (or a region of the RNA) is reverse transcribed into a cDNA molecule and the cDNA is amplified by a DNA polymerase. Although the cDNA is being amplified in this scenario, the HDA target nucleic acid is considered to be the initial RNA because the RNA is present in target bacteria and the reverse-transcribed copy of the RNA (i.e., the cDNA) is what is being amplified by HDA.

### Embodiments

In the following description, numerous specific details are given to provide a thorough understanding of the embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment,” “an embodiment,” or “embodiments” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless indicated otherwise, when a range of any type is disclosed or claimed, it is intended to disclose or claim individually each possible number that such a range could reasonably encompass, including any sub-ranges encompassed therein. Moreover, when a range of values is disclosed or claimed, which Applicant intends to reflect individually each possible number that such a range could reasonably encompass, Applicant also intends for the disclosure of a range to reflect, and be interchangeable with, disclosing any and all sub-ranges and combinations of sub-ranges encompassed therein. Where ranges are given, endpoints are included.

L. monocytogenes Contamination and Environmental Detection

Traditional methods for detection of L. monocytogenes in environmental samples include environmental sample collection followed by enrichment through growth media and finally detection by one of many schemes such as, but not limited to, the following: selective/differential media agar, antibody-based assays (e.g., ELISA and lateral flow assays), molecular methods such as DNA/RNA amplification (e.g., PCR, LAMP, and NEAR) or hybridization assays with nucleic acid probes. For example, traditional methods for L. monocytogenes detection can involve procedures using serial enrichments with increasing selectivity culminating in the isolation of L. monocytogenes on selective-differential agar plates. More recently, PCR and real-time, quantitative PCR (qPCR) have been used for detection in food and environmental samples.

The present disclosure provides methods for rapid detection of L. monocytogenes from environmental samples via detection of L. monocytogenes target nucleic acids. In various embodiments, the methods comprise the steps of providing an environmental sample to be tested; contacting an aliquot of the environmental sample with a lysis mixture under conditions to lyse at least a portion of cells in the aliquot, thereby generating a lysate; contacting an aliquot of the lysate with a detection mixture, thereby generating an assay mixture; in the assay mixture, reverse transcribing target RNA to form target cDNA and amplifying the target cDNA by helicase-dependent amplification (HDA); and detecting presence or absence of the amplified target cDNA, to thereby determine the presence or absence of L. monocytogenes in the environmental sample.

### Environmental Samples

In various embodiments, a sample is provided for purposes of testing for the presence or absence of target bacteria in the sample. The sample can be any sample that may comprise target bacteria. In some embodiments, the provided sample is suspected of being contaminated with bacteria, for example, the target bacteria. In some embodiments, providing a sample to be tested comprises providing a sample to confirm absence of bacterial contamination, for example, absence of the target bacteria. In some embodiments, the target bacteria are of the species L. monocytogenes.

In some embodiments, the presence or absence of target bacteria can be analyzed in a test environmental sample that is derived from food processing and/or beverage processing environmental sources. Non-limiting examples of food processing and/or beverage processing environmental sources include food-handling surface samples (e.g., conveyor belts, blades, cutting surfaces, mixing equipment surfaces, filters, storage containers), room samples (e.g., walls, floors, drains, ventilation equipment), and cleaning equipment (e.g., hoses, cleaning tools).

In preferred embodiments, the sample is an environmental sample. In preferred embodiments, an environmental sample is collected from the environment in a room or a building where foods and/or beverages are produced and/or processed. In some preferred embodiments, environmental samples may be collected from food/beverage production, processing or service sites.

In some embodiments, the environmental sample is from an environment comprising a low concentration of target bacteria cells (e.g., L. monocytogenes cells). As used herein, a “low concentration” of target bacteria cells refers to a concentration that is difficult to detect without amplifying the amount of an indicator associated with the target bacteria (such as by amplifying the amount of cells or the amount of target bacterial nucleic acids). For example, while it is expected that certain environments from food/beverage production, processing or service sites contain no target bacteria like L. monocytogenes, contamination may result in a low concentration of the target bacteria at these sites. Therefore, in some embodiments, the environmental sample is from an environment that can be tested for bacterial contamination of the target bacteria.

In some embodiments, the environment is a surface that is a solid comprising a low concentration of target bacteria cells. In some embodiments, a low concentration of target bacteria is at most 200 colony forming units (CFU) per 1 square inch of a solid surface. In some embodiments, a low concentration of target bacteria is at most 100 CFU per 1 square inch of a solid surface. In some embodiments, a low concentration of target bacteria is at most 50 CFU per 1 square inch of a solid surface. In some embodiments, a low concentration of target bacteria is at most 5 CFU per 1 square inch of a solid surface. In some embodiments, a low concentration of target bacteria is at most 200 CFU per 1 milliliter of a collected sample. In some embodiments, a low concentration of target bacteria is at most 100 CFU per 1 milliliter of a collected sample. In some embodiments, a low concentration of target bacteria is at most 50 CFU per 1 milliliter of a collected sample. In some embodiments, a low concentration of target bacteria is at most 5 CFU per 1 milliliter of a collected sample. In some embodiments, the solid surface comprises from about 10 to about 200 colony forming units (CFU) of target bacteria per 1 square inch of the solid surface. In some embodiments, the solid surface comprises from about 10 to about 100 CFU of target bacteria per 1 square inch of the solid surface. In some embodiments, the solid surface comprises from about 10 to about 50 CFU of target bacteria per 1 square inch of the solid surface. In some embodiments, the solid surface comprises from about 5 to about 200 colony forming units (CFU) of target bacteria per 1 square inch of the solid surface. In some embodiments, the solid surface comprises from about 5 to about 100 CFU of target bacteria per 1 square inch of the solid surface. In some embodiments, the solid surface comprises from about 5 to about 50 CFU of target bacteria per 1 square inch of the solid surface. In some embodiments, the target bacteria are of the species L. monocytogenes.

In various embodiments, samples, such as environmental samples, may be collected with a collection device. In some embodiments, a collection device collects an environmental sample from a surface.

In some embodiments, the collection device is a swab. The swab may be made from various materials, such as, but not limited to, cotton, polyester or polyurethane. In some embodiments, the swab is pre-moistened, such as in solution. The swab may be pre-moistened in buffer or broth. In some embodiments, the swab is pre-moistened in neutralizing buffer, buffered peptone water, or culture medium. In some embodiments, the volume of the pre-moistening liquid on the swab is about 1-10 mL.

In some embodiments, the collection device is a sponge. The sponge may be made from various materials, such as, but not limited to, polyurethane. In some embodiments, a sponge advantageously samples a larger surface area than other sampling devices. In some embodiments, the sponge is pre-moistened. The sponge may be pre-moistened in buffer or broth. In some embodiments, the sponge is pre-moistened in neutralizing buffer, buffered peptone water, or culture medium. In some embodiments, the volume of the pre-moistening liquid on the sponge is about 10-25 mL.

In some embodiments, a sample collection device (e.g., a swab, a sponge) containing sample material may be used to provide an environmental sample according to methods of the present disclosure. In some embodiments, the sample material may be eluted (e.g., rinsed, scraped, expressed) from a sample collection device before using the sample material in a method of the disclosure. In some embodiments, liquid or solid samples may be diluted in a liquid (e.g., water, buffer, broth).

In some embodiments, collecting an environmental sample comprises swabbing with the collection device (e.g., swab or sponge) an area of a solid surface. In some embodiments, the area is about a 1×1 inch area, a 4×4 inch area or a 12×12 inch area. In some embodiments, swabbing comprises swabbing in multiple directions. In some embodiments, the surface is a flat surface. In some embodiments, the environmental sample collected comprises from about 5 to about 200 CFU of target bacteria (e.g., L. monocytogenes). In some embodiments, the environmental sample collected comprises from about 5 to about 100 CFU of target bacteria (e.g., L. monocytogenes). In some embodiments, the environmental sample collected comprises from about 5 to about 50 CFU of target bacteria (e.g., L. monocytogenes). In some embodiments, the environmental sample collected comprises from about 10 to about 200 CFU of target bacteria (e.g., L. monocytogenes).

Methods and kits of the present invention may include a collection device.

Target Nucleic Acids for Detection of L. monocytogenes

Provided by the present disclosure are compositions and methods for detection of target nucleic acids found in target bacteria cells. In various aspects, the target bacteria are L. monocytogenes.

A target nucleic acid may be any type of nucleic acid molecule, such as ribonucleic acid (RNA) or deoxyribonucleic acid (DNA). In some embodiments, the target nucleic acid is an RNA, and is also referred to herein as a “target RNA.”

In some embodiments, a target nucleic acid is abundant in target bacteria, thereby lowering the limit of detection of assays such as those disclosed herein. In some embodiments, a target nucleic acid is present at any or all stages of growth for the target bacteria. For example, the target nucleic acid is present in stationary phase and log phase growth. In some embodiments, the target bacteria are of the species L. monocytogenes. In some embodiments, a target nucleic acid is present at all stages of growth for L. monocytogenes.

In various embodiments, a target nucleic acid of the present disclosure comprises a nucleic acid sequence from bacterial 16S ribosomal RNA (rRNA). The 16S rRNA is a component of the 30S subunit of a prokaryotic ribosome. In some embodiments, a target nucleic acid comprises a sequence from L. monocytogenes 16S rRNA. The 16S rRNA is an RNA molecule present in L. monocytogenes at a high copy number. Without wishing to be bound by theory, targeting 16S rRNA can increase sensitivity due to high copy number within the bacterial cell.

In some embodiments, a target nucleic acid of the present disclosure demonstrates specificity for the target bacteria, for example, L. monocytogenes.

An advantageous property of the target nucleic acids provided by the present disclosure is specificity for the bacteria of interest (e.g., L. monocytogenes), as demonstrated by examples provided herein. Additionally, the present disclosure demonstrates that these target nucleic acids are accessible for detection, for example, by using compositions and methods provided herein.

Forward and reverse primers as well as probes for the target nucleic acid of the present disclosure can be designed. For example, primers according to the present disclosure can be used to reverse transcribe a target RNA to form a corresponding target cDNA. As used herein, a “corresponding target cDNA” or simply “target cDNA” is a cDNA that is generated by reverse transcribing a target RNA. Additionally, in some embodiments, primers can be used to amplify a target cDNA. In some embodiments, a single pair of one forward primer and one reverse primer can be used to reverse transcribe a target RNA to form a corresponding target cDNA and to amplify the target cDNA. In some embodiments, one forward primer and two reverse primers can be used, such that a first reverse primer initiates transcription of a target RNA to form a corresponding target cDNA and a second reverse primer can be used to amplify the target cDNA. In some embodiments, the first reverse primer is an unblocked primer and the second reverse primer is a blocked primer. In some embodiments, the forward primer is a blocked primer.

In some embodiments, blocked primers are used in an assay of the present disclosure. In some embodiments, a forward primer, a reverse primer, or both are blocked primers. In some embodiments, a ribobase in both forward and reverse primers sits across from single nucleotide polymorphism (SNP) in Listeria species. Without wishing to be bound by any theory, RNaseHII is very sensitive to mismatches and will cut a ribobase that has high complementarity to the base in the target sequence. Cutting activates the primer for polymerization.

In some embodiments, an unblocked (also referred to herein as “non-blocked”) primer is also used in an assay of the present disclosure. Blocked primers do not polymerize against RNA targets, therefore the reverse blocked primer cannot initiate first-strand synthesis on the L. monocytogenes 16S rRNA. A non-blocked reverse primer that is only specific to Listeria species is used to initiate the reverse transcriptase reaction in an assay of the present disclosure. In some embodiments, this non-blocked primer is at a lower concentration and amplifies both exclusive and inclusive Listeria rRNA. Without wishing to be bound by any theory, the blocked reverse primer then continues amplification on the cDNA made by the reverse transcriptase, but amplifies only L. monocytogenes cDNA.

In some embodiments, L. monocytogenes detection may include use of a blocked forward primer, a blocked reverse primer and an unblocked reverse primer. In some embodiments, L. monocytogenes detection may include use of primers shown in Table 1A. The blocking group of the blocked primers in Table 1A comprises rDXXDM, wherein R is an RNA residue, D is a DNA residue, M is a mismatched residue and X is a spacer. The mismatched residue (M) is underlined.

TABLE 1A
Primers

| | Primer | | |
|---|---|---|---|
| SEQ ID NO. | Name | Sequence 5′-3′ | Description |
| 1 (+2 Spacers, | eF2 | GGC TAC ACA CGT GCT ACA ATG GAT | Blocked Forward |
| each iSPC3, | | [rA]G/iSpC3//iSpC3/TG | Primer (Blocking |
| plus thymine | | | group rDXXDM) |
| and guanine) | | | |
| 2 (+2 Spacers, | R3 | GCA GCC TAC AAT CCG AAC TGA GAA T[rA]G/ | Blocked Reverse |
| each iSPC3, | | iSpC3//iSpc3/TC | Primer (Blocking |
| plus thymine | | | group rDXXDM) |
| and cytosine) | | | |
| 3 | R12 | CGA GTT GCA GCC TAC AAT CCG AAC TGA G | Unblocked |
| | | | Reverse Primer |

In some embodiments, L. monocytogenes detection may include use of a primer comprising the nucleic acid sequence of 5′ GGC TAC ACA CGT GCT ACA ATG GAT [rA]G 3′(SEQ ID NO: 1), a primer comprising the nucleic acid sequence of 5′ GCA GCC TAC AAT CCG AAC TGA GAA T [rA]G 3′ (SEQ ID NO: 2), wherein the “r” denotes that the base is RNA, and a primer comprising the nucleic acid sequence of 5′ CGA GTT GCA GCC TAC AAT CCG AAC TGA G 3′ (SEQ ID NO: 3).

Also provided herein is a primer comprising or consisting of the nucleic acid sequence of GGC TAC ACA CGT GCT ACA ATG GAT [rA]G (SEQ ID NO: 1), wherein the “r” denotes that the base is RNA, or a sequence with at least 90%, at least 95%, at least 96%, at least 97%, at least 98% or at least 99% sequence identity thereto. Also provided herein is a primer comprising or consisting of the nucleic acid sequence of GCA GCC TAC AAT CCG AAC TGA GAA T [rA]G (SEQ ID NO: 2), wherein the “r” denotes that the base is RNA, or a sequence with at least 90%, at least 95%, at least 96%, at least 97%, at least 98% or at least 99% sequence identity thereto. Also provided herein is a primer comprising or consisting of the nucleic acid sequence of CGA GTT GCA GCC TAC AAT CCG AAC TGA G (SEQ ID NO: 3) or a sequence with at least 90%, at least 95%, at least 96%, at least 97%, at least 98% or at least 99% sequence identity thereto. Also provided are compositions and kits comprising one or more of any of these primers.

### Bacterial Cell Lysis

Methods of the present disclosure include cell lysis to release molecules, including nucleic acids such as DNA and RNA, from cells. Following release of nucleic acids, additional methods may be employed for processing the nucleic acids, including reverse transcription, amplification and/or detection of nucleic acids. The provided lysis methods include advantages over other lysis methods, such advantages including ease of use, lytic efficiency and non-inhibitory properties to downstream methods such as helicase-dependent amplification (HDA).

Provided herein are compositions and methods to lyse bacterial cells. These compositions and methods may be used for lysing gram-negative bacteria and/or gram-positive bacteria. In some embodiments, cell lysis compositions and methods of the disclosure are used for lysing gram-positive bacteria, such as Listeria. In some embodiments, cell lysis compositions and methods of the disclosure are used for lysing both gram-negative bacteria and gram-positive bacteria.

In some embodiments, cell lysis according to the present disclosure comprises use of a lysis mixture comprising lysis mixture components. In some embodiments, the set of lysis mixture components comprises: (i) at least one lytic enzyme (for example, lysozyme and/or mutanolysin); (ii) at least one protease or enzyme that degrades protein (for example, proteinase K and/or achromopeptidase); (iii) a chelating resin; and (iv) a pH buffer (for example, Tris). In some embodiments, the chelating resin is Chelex®-100 resin (also referred to herein as simply “Chelex-100” or “Chelex”), which is identifiable by CAS number 11139-85-8. Chelex-100 is a styrene divinylbenzene copolymer containing paired iminodiacetate ions. Chelex-100 is an insoluble resin that chelates metals and divalent cations, and is known for lysing bacterial cells. In some embodiments, the pH buffer should be selected to produce an optimal pH for a downstream HDA method. In some embodiments, an optimal pH is a pH of about 8.8. Thus, the same lysis mixture described by the present disclosure can be used for gram-negative and gram-positive bacteria.

In some embodiments, the set of lysis mixture components comprises one or more of: (i) lysozyme; (ii) mutanolysin; (iii) proteinase K; (iv) Chelex®-100; and (v) Tris buffer. In some embodiments, the set of lysis mixture components comprises: (i) lysozyme; (ii) mutanolysin; (iii) proteinase K; (iv) Chelex®-100; and (v) Tris buffer. In some embodiments, the set of lysis mixture components further comprises sucrose. In some embodiments, the set of lysis mixture components further comprises dextran.

In some embodiments, the set of lysis mixture components comprises one or more of: (i) lysozyme; (ii) mutanolysin; (iii) proteinase K; (iv) achromopeptidase; (v) Chelex®-100; and (vi) Tris buffer. In some embodiments, the set of lysis mixture components comprises: (i) lysozyme; (ii) mutanolysin; (iii) proteinase K; (iv) achromopeptidase; (v) Chelex®-100; and (vi) Tris buffer. In some embodiments, the set of lysis mixture components further comprises sucrose. In some embodiments, the set of lysis mixture components further comprises dextran.

In some embodiments, the lysis mixture is lyophilized. In some embodiments, one or more components of the lysis mixture are lyophilized together as one lyophilized pellet. In some embodiments, one or more components of the lysis mixture are lyophilized separately as more than one lyophilized pellet. For example, a first set of lysis mixture components comprising at least one lytic enzyme (e.g., lysozyme and/or mutanolysin) and at least one protease or enzyme that degrades protein (for example, proteinase K and/or achromopeptidase) may be lyophilized as a first pellet and a second set of lysis mixture components comprising a chelating resin (e.g., Chelex-100) may be lyophilized as a second pellet.

In some embodiments, one or more lyophilized pellets, each comprising one or more components of a lysis mixture are resuspended. For example, the one or more lyophilized pellets may be resuspended with a liquid composition. In some embodiments, the one or more lyophilized pellets may be resuspended with a sample or solution comprising target bacteria cells. In some embodiments, the one or more lyophilized pellets may be resuspended with an aliquot of a sample that has been pre-treated according to pre-treatment methods described above. In some embodiments, the concentration of one or more of the components of the lysis mixture after resuspension is in the range of concentrations listed in Table 2. In some embodiments, the set of lysis mixture components comprises the components listed in Table 2 and the concentration of each of the listed components after resuspension of the lyophilized lysis mixture components is in the range specified in Table 2. In some embodiments, the set of lysis mixture components comprises one or more of the components listed in Table 3 and the concentration of each component after resuspension of the lyophilized lysis mixture components is the concentration listed in Table 3. In some embodiments, the set of lysis mixture components comprises the components listed in Table 3 and the concentration of each of the listed components after resuspension of the lyophilized lysis mixture components is the concentration listed in Table 3.

TABLE 2
Concentration ranges for lysis mixture components

| Component | Concentration range | |
|---|---|---|
| Tris HCl pH 8.8 | 5-50 | mM |
| Chelex-100 | 0-7.5% | (m/v) |
| Sucrose | 2-5% | (m/v) |
| Lysozyme | 0-1 | mg/mL |
| Proteinase K | 0-1 | mg/mL |
| Mutanolysin | 0-30 | Units/mL |
| EGTA | 0-5 | mM |
| Achromopeptidase | 0-150 | Units/mL |
| Dextran | 0-2% | (m/v) |

TABLE 3
Lysis mixture components and concentrations

| Component | Concentration | |
|---|---|---|
| Tris HCl pH 8.8 | 5.7 | mM |
| Chelex-100 | 4.5% | (m/v) |
| Sucrose | 3% | (m/v) |
| Lysozyme | 0.8 | mg/mL |
| Proteinase K | 0.8 | mg/mL |
| Mutanolysin | 20 | Units/mL |
| EGTA | 2.6 | mM |
| Achromopeptidase | 85.6 | Units/mL |
| Dextran | 0.56% | (m/v) |

In some embodiments, a lysis mixture may also be referred to herein as a “lysis buffer”.

In some embodiments, a method of the present disclosure comprises contacting an aliquot of a provided sample (e.g., environmental sample) with a lysis mixture (or lysis mixture components) under conditions to lyse at least a portion of cells in the aliquot, thereby generating a lysate.

In some embodiments, the entire collected sample is subjected to the lysis procedure, which is conducted in a small volume of lysis buffer.

In some embodiments, the provided lysis compositions and methods can be used with downstream methods such as reverse transcription, helicase-dependent amplification (HDA), and/or nucleic acid detection methods.

In some embodiments, the present disclosure provides cell lysis compositions and methods that can be used with downstream methods for detection of target nucleic acids. In some embodiments, the provided lysis compositions and methods can be used with downstream methods for determining the presence or absence of target bacteria in an environmental sample.

In some embodiments, following cell lysis, some or a portion of a sample comprising lysed cells (i.e., the lysate) is used in a downstream method, such as HDA.

Among other things, the lysis methods of the present disclosure provide the advantages of improved limit of detection and improved sensitivity of downstream assays that measure target nucleic acid presence and/or levels as compared to other lysis methods.

Other lysis methods include lysis protocols for samples that are target-rich. For instance, other methods use clinical samples, such as throat swabs of patients, which contain many bacteria. Target-rich samples allow use of lysis methods that have low lytic efficiency, such as lysis in which only 10% of bacteria in a sample is lysed and yet still enable detection of the target with an assay due to the high amount of bacteria. By contrast, the present lysis method enables lysis of more bacterial cells. In some embodiments, lysis methods of the present disclosure enable lysis of at least 90% of cells in a sample. In some embodiments, lysis methods of the present disclosure enable lysis of nearly every cell in the sample. In some embodiments, the lysis method of the present disclosure enables a limit of detection of approximately 10-100 cells with downstream assays.

Another improvement of the present lysis methods provides for the elimination of harsh chemicals that need to be neutralized or diluted prior to a sample being added to or used for a downstream method, such as a detection assay. While other lysis methods use such harsh chemicals, the lysis method disclosed herein does not use chemicals that require neutralization or dilution prior to target detection. The present lysis method thus improves workflow by minimizing touchpoints and minimizing steps from sample collection through analysis.

In some embodiments, a mechanical lysis procedure may be used to lyse cells. In some embodiments, a mechanical lysis procedure comprises use of beads. In some embodiments, the beads are Zirconia Silicate beads. In some embodiments, the beads are glass beads. In some embodiments, the bead diameter is between 0.1 mm and 0.5 mm. In some embodiments, the bead diameter is 0.1 mm. In some embodiments, the bead diameter is 0.5 mm. In some embodiments, a mechanical lysis procedure comprises use of sharp particles. In some embodiments, the sharp particles are garnet sharp particles. In some embodiments, the sharp particles are 0.3 mm garnet sharp particles.

In some embodiments, a sample or aliquot of a sample to be tested for a target bacteria is combine with beads or sharp particles for mechanical lysis of cells in the sample or aliquot of the sample. In some embodiments, a sample or aliquot of a sample to be tested for a target bacteria is resuspended in a buffer and combined with beads or sharp particles for mechanical lysis. In some embodiments, the buffer is Buffered Peptone Buffer. In some embodiments, the buffer is TE buffer. In some embodiments, the buffer is PT buffer. In some embodiments, a sample or aliquot of a sample to be tested for a target bacteria is resuspended in molecular-grade water and combined with beads or sharp particles for mechanical lysis.

In some embodiments, the volume of a sample, aliquot of a sample, or resuspended sample or aliquot of a sample that is combined with beads or sharp particles is between 50 μL to 500 μL. In some embodiments, the volume of a sample, aliquot of a sample, or resuspended sample or aliquot of a sample that is combined with beads or sharp particles is 200 μL. In some embodiments, the bead mass that is combined with the sample, aliquot of a sample, or resuspended sample or aliquot of a sample is 25 mg, 50 mg, 75 mg, or 100 mg. In some embodiments, the bead mass that is combined is 50 mg. In some embodiments, 200 μL of a sample, aliquot of a sample, or resuspended sample or aliquot of a sample is combined with 50 mg of beads.

In some embodiments, lysis comprises disrupting the beads in a disruptor. In some embodiments, lysis comprises disrupting the beads in a disruptor for a time ranging from about 1 to about 10 minutes. In some embodiments, the disruption time is about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some embodiments, the disruption time is about 2 minutes. In some embodiments, the disruption time is about 5 minutes.

### Helicase-Dependent Amplification

Helicase-dependent amplification (HDA), is a method for nucleic acid amplification that mimics an in vivo process of DNA replication, using helicase(s) to isothermally unwind nucleic acid duplexes. The resulting separated strands of the nucleic acid duplex provide templates for nucleic acid amplification. The platform technology for HDA is described in U.S. Pat. No. 7,282,328, “Helicase dependent amplification of nucleic acids,” which is incorporated by reference herein in its entirety.

Unlike other approaches for in vitro amplification of nucleic acids that use heat to separate nucleic acid duplexes, HDA uses one or more helicases. The separated nucleic acid strands serve as single-stranded templates for in vitro amplification of nucleic acids. Sequence-specific primers hybridize to the templates and are then extended by DNA polymerases to amplify an HDA target nucleic acid. This process repeats itself so that exponential amplification can be achieved at a single temperature.

A diversity of helicases can be used for HDA. Non-limiting examples of helicases for HDA are described in U.S. Pat. Nos. 7,282,328 and 7,662,594. For example, a thermostable helicase, which is a helicase that is capable of unwinding double-stranded DNA under elevated temperatures (e.g., preferred reaction temperature above about 60° C.), can be used in HDA. Examples of thermostable helicases include UvrD-like helicases. For example, Tte UvrD helicase is a helicase from the thermophilic organism

Thermoanaerobacter tengcongensis.

In various aspects of the present disclosure, a helicase selected for use in HDA methodology is a thermostable helicase. In some embodiments, the helicase is a Tte-UvrD helicase.

Regions of nucleic acid strands that have been separated by one or more helicases can be amplified as part of HDA methodology. One or more polymerases are used for amplification. If the nucleic acid to be amplified is DNA, a DNA polymerase can be used for amplification. When the initial nucleic acid provided for an HDA method is RNA, a reverse transcriptase is used to first copy the RNA (or a region of the RNA) into a cDNA molecule and the cDNA is amplified by a DNA polymerase.

The DNA polymerase acts on the HDA target nucleic acid to extend the primers hybridized to the nucleic acid templates in the presence of dNTPs to form primer extension products complementary to the nucleotide sequence on the nucleic acid template.

DNA polymerases for HDA may be selected from polymerases lacking 5′ to 3′ exonuclease activity and which additionally may optionally lack 3′-5′ exonuclease activity. In some embodiments, the polymerase used according to the present disclosure is a thermostable polymerase. In some embodiments, the polymerase is Gst Polymerase. In some embodiments, the polymerase is WarmStart Gst polymerase, which is a Gst polymerase that has been modified to function at about 45° C. or higher. In some embodiments, the polymerase is Bst polymerase. In some embodiments, the polymerase is WarmStart Bst polymerase, which is a Bst polymerase that has been modified to function at about 45° C. or higher.

In some embodiments, HDA methods of the disclosure are also referred to as “thermostable helicase-dependent amplification” (tHDA). As used herein, tHDA is a type of HDA that uses a thermostable helicase and a thermostable polymerase. The thermostable properties of the helicase and polymerase enable performing HDA at high temperatures (e.g., 45° C.-75° C.), which may increase the specificity of target nucleic acid amplification.

Generally, primers suitable for use in HDA are short synthetic oligonucleotides, for example, having a length of more than 10 nucleotides and less than 50 nucleotides. Oligonucleotide primer design involves various parameters such as string-based alignment scores, melting temperature, primer length and GC content (Kampke et al., Bioinformatics 17:214-225 (2003)). When designing a primer, one of the important factors is to choose a sequence within the target fragment which is specific to the nucleic acid molecule to be amplified. Another important factor is to decide the melting temperature of a primer for HDA reaction. The melting temperature of a primer is determined by the length and GC content of that oligonucleotide. Preferably, the melting temperature of a primer is about equal to 10° C. higher than the temperature at which the hybridization and amplification will take place. For example, if the temperature of the hybridization and amplification is 60° C., the melting temperature of a pair of primers designed for that reaction should be in a range between 6° C. and 75° C. In preferred embodiments, the melting temperature of primers according to the present disclosure is about 65° C.

Each primer hybridizes to each end of the HDA target nucleic acid and may be extended in a 5′ to 3′ direction by a polymerase using the target nucleotide sequence (or complementary sequence) as a template. To achieve specific amplification, a homologous or perfect match primer is preferred. However, primers may include sequences at the 5′ end which are non-complementary to the target nucleotide sequence(s). Alternatively, primers may contain nucleotides or sequences throughout that are not exactly complementary to the HDA target nucleic acid. Primers may represent analogous primers or may be non-specific or universal primers for use in HDA as long as specific hybridization can be achieved by the primer-template binding at a predetermined temperature.

HDA methods may include more than one pair of primers. HDA methods using more than one pair of primers may be used to amplify nucleic acids comprising different target sequences of HDA.

In some embodiments, HDA methods include use of three primers. In some embodiments, one forward primer and two reverse primers can be used, such that a first reverse primer initiates transcription of a target RNA to form a corresponding target cDNA and a second reverse primer can be used to amplify the target cDNA. In some embodiments, the first reverse primer is an unblocked primer and the second reverse primer is a blocked primer. In some embodiments, the forward primer is a blocked primer. In some embodiments, L. monocytogenes detection according to HDA methods may include use of a blocked forward primer, a blocked reverse primer and an unblocked reverse primer. In some embodiments, L. monocytogenes detection may include use of primers shown in Table 1A.

In addition to helicase(s), polymerases, and primers, HDA methods may also use single stranded binding proteins (SSB). Some helicases show improved activity in the presence of SSB, which can stabilize unwound single-stranded nucleic acids so that they do not re-anneal. In some embodiments where a thermostable helicase is used, the presence of a single stranded binding protein is optional.

HDA methods may also use one or more accessory proteins. The term “accessory protein” refers to any protein capable of stimulating helicase activity. For example, E. coli MutL protein is an accessory protein for enhancing UvrD helicase melting activity. In some embodiments, accessory proteins are desirable for use with selected helicases. In alternative embodiments, unwinding of nucleic acids may be achieved by helicases in the absence of accessory proteins.

Other components that may be used in HDA include one or more buffers, one or more chemical reagents, one or more small molecules, salts (e.g., MgSO₄, KCl and NaCl), additives, and/or excipients. Examples of additives include Dithiothreitol (DTT) and Tween-20. Examples of excipients include sucrose, dextran and BSA.

In some embodiments, components for HDA comprise one or more buffers, salts (e.g., MgSO₄, KCl and NaCl), additives, and/or excipients. Examples of additives include dithiothreitol (DTT) and Tween-20. Examples of excipients include sucrose, dextran and BSA. Sucrose, dextran and BSA are inert components for lyophilization.

In various embodiments of the present disclosure, HDA occurs in the presence of a set of components comprising a helicase, an energy source, DNA polymerase, deoxynucleotide triphosphate (dNTPs) and primers. Examples of an energy source are nucleotide triphosphates (NTPs) or dNTPs. In some embodiments, the set of components further comprise a single stranded binding protein. An example of a single stranded binding protein is the thermophilic archaeal Sulfolobus solfataricus SSB (SSo-SSB). In some embodiments, the set of components further comprise a Tris buffer, MgSO₄, KCl, NaCl, DTT, Tween-20, sucrose, dextran, and BSA.

In various embodiments of the present disclosure, reverse transcription of RNA is combined with amplification of the resulting cDNA via HDA. For example, an initial target RNA in a provided sample is reverse transcribed to form target cDNA, and the target cDNA is amplified by HDA. An illustrative non-limiting example of a method that combines reverse transcription of RNA with amplification of the resulting cDNA via HDA is shown in FIG. 17 of U.S. Pat. No. 7,662,594. Methods of the present disclosure that combine reverse transcription and HDA include a reaction or a series of reactions that comprise reverse transcription, helicase-dependent denaturation and amplification. Either a reverse transcriptase or a polymerase with reverse transcription properties can be used to synthesize cDNA by reverse transcription of target RNA. Examples of reverse transcriptases include mutants of or wild-type Moloney Murine Leukemia Virus (MMLV) reverse transcriptase and mutants of or wild-type Avian Myeloblastosis Virus (AMV) reverse transcriptase. In some embodiments, a reverse transcriptase used according to the present disclosure is a mutant of the MMLV reverse transcriptase referred to as NxtScript Reverse Transcriptase (Roche Custom Biotech).

In some embodiments, methods for detecting target bacteria provided herein combine reverse transcription of a target RNA with amplification of the resulting cDNA. In various embodiments, reverse transcription and DNA amplification occur at the same time and in the same reaction vessel. A first strand cDNA is synthesized by reverse transcription of the target RNA, forming a DNA/RNA duplex. A helicase unwinds the DNA/RNA duplex into at least partial single strand nucleic acids and a SSB stabilizes the single strand nucleic acids. The single-stranded RNA enters a next round of reverse transcription (RT) reaction, generating more first strand cDNA. The single-stranded DNA is converted into double-stranded DNA by DNA polymerase and amplified concurrently in the HDA reaction. This process repeats itself to achieve exponential amplification of the RNA target sequence.

In various embodiments comprising reverse transcription of RNA combined with amplification of the resulting cDNA via HDA, sequence-specific primers are used for the reverse transcription and amplification reactions. In some embodiments, three sequence-specific primers are used: one unblocked primer hybridizes to the 3′ end of the target nucleic acid (e.g., the target RNA, such as the 16S rRNA); a first blocked primer hybridizes to the 3′ end of the complimentary strand which is produced by reverse transcription (e.g., the target cDNA); and a second blocked primer hybridizes to the 3′ end of the strand that is produced by amplification initiated by the first blocked primer (e.g., a ssDNA formed by unwinding the duplex formed during a stage of target cDNA amplification). Subsequently, the blocked primers hybridize to the analogous strands in the amplified products for continued rounds of amplification.

In some embodiments, primers selected for HDA methods of the present disclosure are the primers described above (e.g., Table 1A) and in Examples 1-2.

In some embodiments, reverse transcription and HDA reactions occur in a single reaction vessel with a single buffer, such that cDNA copies of the RNA target sequence act as a template for DNA amplification at the same time as more cDNA is generated from RNA by reverse transcription. Helicases that unwind both RNA-DNA duplexes and DNA duplexes are preferred in reactions that occur in a single reaction vessel. Such a helicase can be, for example, Tte-UvrD helicase. In some embodiments, reverse transcription and amplification are performed isothermally. An advantage of these embodiments is that unwinding by helicase and amplification can effectively occur at a single temperature. Methods of the present disclosure that combine reverse transcription and HDA can be used to detect and/or quantify target bacteria using HDA methodology.

In some embodiments, reverse transcription and HDA occur in the presence of a set of components comprising a helicase, an energy source, DNA polymerase, reverse transcriptase, dNTPs and primers. In some embodiments, the set of components further comprise a single stranded binding protein. In some embodiments, the set of components further comprise a Tris buffer, MgSO₄, KCl, NaCl, DTT, Tween-20, sucrose, dextran, and BSA.

### Amplicon Contamination Control for Helicase-Dependent Amplification (HDA)

In some embodiments of the present disclosure, HDA compositions (e.g., mixtures comprising HDA components) and/or HDA methods are modified to incorporate components and/or methods for amplicon control. As used herein, the term “amplicon control” refers to reducing or eliminating carryover contamination. Amplicon control helps prevent prior positive reactions from contaminating and triggering false positives on subsequent negative reactions.

In some embodiments, amplicon control involves a reaction that occurs before reverse transcription and amplification reactions described herein. Such an amplicon control reaction destroys or removes contamination amplicon. In some embodiments, an amplicon control reaction involves at least two enzymes that enable destruction of contamination amplicon.

In some embodiments, an amplicon control reaction occurs in the same tube as HDA. In some embodiments, an amplicon control reaction must not occur during DNA amplification as it would destroy any amplified target (amplicon).

In some embodiments, an amplicon control reaction occurs at a different temperature as HDA. In some embodiments, an amplicon control reaction requires a temperature of about 37° C. In some embodiments, one or more enzymes involved in amplicon control have to be inactivated at the reverse transcription and amplification step of HDA (about 65° C.). In some embodiments, at least one enzyme involved in amplicon control is inactivated at temperatures higher than about 50° C.

In some embodiments, components for amplicon control comprise: (i) an enzyme that binds uracil in a DNA strand and converts it into an apurinic site; (ii) an enzyme that cleaves DNA at apurinic sites; and (iii) a specialized dNTP that is recognized by the enzyme of (i).

In some embodiments, components for amplicon control comprise: (i) uracil DNA glycosylase (also referred to as “UDG” or “UNG”); (ii) Endonuclease VIII; and (iii) dUTP. In some embodiments, the UDG is a thermolabile UDG. A non-limiting example of a UDG that may be used as described herein is Antarctic Thermolabile UDG enzyme (New England Biolabs, Ipswich, MA).

In some embodiments, dUTP is incorporated into all amplicons in an HDA method by Gst polymerase. As disclosed herein, the inclusion of dUTP does not inhibit Gst polymerase or the reverse transcriptase. The concentration of dUTP should be optimized to sufficiently remove unwanted amplicon without slowing the reaction significantly.

Detection and/or Quantification of Target Nucleic Acids

Various methods and instruments can be used to detect and/or quantify target nucleic acids in conjunction with other methods of the present disclosure. As used herein, “to detect” means to identify the presence or absence of the target nucleic acids and “to quantify” means to measure or calculate the quantity of the target nucleic acids. In some embodiments, detection and quantification occur concomitantly. In some embodiments, detection and quantification are achieved by the same means.

In various embodiments, detection and/or quantification comprises identifying and/or measuring amplified nucleic acids, for example, the amplicon products of HDA.

Amplified nucleic acid products may be identified and/or measured by methods including ethidium-bromide staining or by means of a label selected from the group consisting of a radiolabel, a fluorescent label, and an enzyme.

Fluorescence measurement is a type of detection/quantification method that can be used for detection and/or quantification of amplified nucleic acids of the present disclosure. For example a fluorescent intercalator that is only fluorescent when bound to dsDNA, may be used for fluorescence measurement. Alternatively, fluorescent probes may be designed to only fluoresce when bound to specific nucleic acid sequences, rather than any dsDNA. In some embodiments, amplified target nucleic acids can be detected/quantified using quenched fluorescent oligonucleotides that generate fluorescence when bound to or incorporated into an amplification product. Fluorescence can be measured using an instrument called a fluorometer.

Real-time measurement of nucleic acid amplification is also a type of detection/quantification method. In real-time measurement, the progress of nucleic acid amplification is monitored as it occurs (i.e., in real time). Measurements are therefore collected throughout the process of amplification, rather than at the end of amplification. Many real-time methods use fluorescence as the readout for nucleic acid amplification. Real-time reactions are characterized by the point in time during cycling when amplification of a target is first detected rather than the amount of target accumulated after a fixed number of amplification cycles. The higher the starting copy number of the nucleic acid target, the sooner a significant increase in signal (e.g., fluorescence) is observed. In contrast, an endpoint assay measures the amount of accumulated product at the end of the amplification. In various methods of the present disclosure, real-time measurement of nucleic acid amplification is used for detection and/or quantification of a target nucleic acid. In some embodiments, real-time measurement is used to detect the amplicon products of HDA methods of the disclosure. In some embodiments, the step of detecting according to a method of the present disclosure comprises real-time measurement of amplified target cDNA.

In various embodiments, detection and/or quantification comprises identifying and/or measuring the amplicon products of HDA. In some embodiments, detection and/or quantification of the amplicon products of HDA comprises subjecting the assay mixture to successive cycles of amplification to generate a signal from a probe designed to detect the amplicon (e.g., a fluorescent signal) and quantifying the nucleic acid presence. In some embodiments, quantifying the nucleic acid presence is based on the signal cycle threshold of the amplification reaction. In some embodiments, the step of detecting according to a method of the present disclosure comprises quantifying the presence of amplified target cDNA based on a fluorescence signal cycle threshold of the amplification reaction. In some embodiments, quantifying the nucleic acid presence is based on the strength of a detection signal.

In some embodiments, the step of detecting presence or absence of amplified target cDNA according to a method of the present disclosure comprises determining fluorescence signal from a fluorescent molecular probe as an indication of presence based on the speed of the signal. In some embodiments, the step of detecting further comprises determining fluorescence signal from the probe as an indication of presence based on the rate at which signal increases and the strength of the signal. In some embodiments, the step of detecting presence or absence of the amplified target cDNA comprises: (i) detecting the speed of the signal and the strength of the signal that meets a threshold speed and strength and thereby determining the presence of the target bacteria in a sample (e.g., environmental sample); or (ii) detecting that the speed of the signal and the strength of the signal does not meet a threshold speed and strength and thereby determining the absence of the target bacteria in the sample.

In some embodiments, an algorithm is used for detection and/or quantification. As a non-limiting example for a molecular probe (e.g., a fluorescent molecular probe), an algorithm can be used to determine signal from the probe as an indication of presence based on the speed of the signal, as determined by the Cq (when fluorescence intensity exceeds a noise threshold) and the slope (the rate at which fluorescence increases), and the strength of the signal (maximum fluorescence). If the generated signal meets the threshold speed and strength criteria, it will be called “present” (positive signal). If it does not meet these criteria, it will be called as “absent” (negative signal). In some embodiments, the speed of the signal and the strength of the signal is considered to meet the threshold speed and strength criteria if it matches or surpasses the threshold values set for speed and strength. In some embodiments, a speed signal is considered to match or surpass the threshold value set for speed if it exceeds a noise threshold at or before the speed threshold value. In some embodiments, a strength signal is considered to match or surpass the threshold value set for strength if the maximum fluorescence matches or surpasses the strength threshold value.

In some embodiments, the presence and absence assay signal are reliant on the limit of detection of the assay. Samples with a target concentration (for example, the concentration of L. monocytogenes rRNA prior to reverse transcription and amplification) greater than the limit of detection will have a strong fluorescent signal with a small Cq (fast onset) and will be called as present. However, for samples with target concentrations close to the limit of detection, an absence signal does not mean an absolute absence of the target. Usually multiple replicates are necessary to detect presence at these levels. For samples with a much lower target concentration than the limit of detection of the assay, the amount of target present in the sample will not be enough to generate a fast and a strong fluorescent signal and will fail to meet the criteria for making a positive call, therefore, will be called as absent/negative.

Amplification assay results may be read by an automated reader, such as a reader that measures fluorescence. Alternatively or additionally, assay results may be detected by enzymatic detection methods or gel electrophoresis.

In some embodiments, detection of amplified nucleic acids comprises quantification of the amplified product (e.g., quantification of the amplicon). In some embodiments, quantification comprises measuring relative levels of a readout, such as relative levels of a test readout (e.g., a readout indicating presence of a target nucleic acid) relative to a control readout. In some embodiments, the readout is a fluorescence readout.

In some embodiments, detection of amplified nucleic acids comprises detecting presence or absence of the amplified product.

In some embodiments, the amplicon comprises the nucleic acid sequence (read in the 5′ to 3′ direction) of: GGCTACACACGTGCTACAATGGATAGTACAAAGGGTCGCGAAGCCGCGAGGTGG AGCTAATCCCATAAAACTATTCTCAGTTCGGATTGTAGGC (SEQ ID NO: 4). In some embodiments, the amplicon comprises a nucleic acid sequence having at least 90%, 91%, 92%, 93%, 94%, 95%, 96%, 97%, 98%, or 99% but less than 100% sequence identity to SEQ ID NO: 4. In some embodiments, the amplicon consists of the nucleic acid sequence of SEQ ID NO: 4.

In some embodiments, one or more target nucleic acids are amplified according to HDA methods and the amplified nucleic acid is detected using a probe designed to identify the amplified nucleic acid. In some embodiments, an initial target RNA present in a provided sample is reverse transcribed to form target cDNA, the target cDNA is amplified by HDA, and the amplified target cDNA is detected.

In some embodiments, the probe used to detect amplified nucleic acids of the present disclosure is a fluorescent hybridization probe. In some embodiments, the fluorescent hybridization probe is a conditional fluorescent hybridization probe that emits fluorescence when hybridized to a nucleic acid molecule. In some embodiments, a conditional fluorescent hybridization probe comprises a fluorophore and a quencher that prevents the fluorophore from generating fluorescence unless the probe is bound to the amplified nucleic acid being detected.

In some embodiments, the probe used to detect amplified nucleic acids of the present disclosure comprises a ribobase to bind a L. monocytogenes-specific SNP. In some embodiments, the ribobase that binds to a L. monocytogenes-specific SNP increases the selectivity of the assay with several exclusive Listeria species including Listeria riparia, Listeria grayi and Listeria booriae.

In some embodiments, the conditional fluorescent hybridization probe comprises the nucleic acid sequence of: CCT CGC GG[rC] TTC GC (SEQ ID NO: 5), wherein the “r” denotes that the base is RNA. In some embodiments, the conditional fluorescent hybridization probe comprises a nucleic acid sequence with at least 90% sequence identity to SEQ ID NO: 5. In some embodiments, a fluorophore may be linked at either the 5′ or 3′ end of the probe. In some embodiments, a quencher may be linked at the opposite end of the probe to that of the fluorophore.

In some embodiments, the conditional fluorescent hybridization probe comprises 5′/56-ROXN/CCT CGC GG[rC] TTC GC/3IABkFQ 3′ (SEQ ID NO: 5; also referred to by the present disclosure as “Probe 7 ROX”), wherein “56-ROXN” is ROX (carboxy-X-rhodamine) fluorophore; “3IABkFQ” is Iowa Black FQ Quencher; and the “r” denotes that the base is RNA. This probe comprises DNA bases and a single RNA base. When the probe binds the nucleic acid being detected, the RNA base can be recognized by an enzyme (e.g., RNase H2) that cleaves the probe, thereby separating the quencher and the fluorophore and generating fluorescence.

In some embodiments, detection of amplified nucleic acid occurs in the same reaction vessel as nucleic acid amplification. In some embodiments, detection of amplified nucleic acid occurs in the same reaction vessel as reverse transcription of an initial target RNA to form target cDNA and amplification by HDA of the target cDNA; in this scenario, the nucleic acid being detected is the amplified cDNA. In some embodiments, the amplified cDNA is detected/quantified using an instrument that supports isothermal DNA/RNA amplification methods. In some embodiments, the instrument takes fluorescence measurements in real-time. As non-limiting examples, the instrument may be a Genie® II or Genie® III reader (made by OptiGene, UK). In some embodiments, the instrument used according to methods of the present disclosure is a Genie® II reader (OptiGene, UK).

In some embodiments, detection of amplified nucleic acid comprises use of a detection mixture comprising a set of detection mixture components. In some embodiments, the set of detection mixture components comprises a probe. In some embodiments, the probe is a conditional fluorescent hybridization probe. In some embodiments, the conditional fluorescent hybridization probe emits fluorescence when hybridized to a nucleic acid molecule comprising the nucleic acid sequence of: GCGAAGCCGCGAGG (SEQ ID NO: 6). In some embodiments, the conditional fluorescent hybridization probe emits fluorescence when hybridized to a nucleic acid molecule comprising the nucleic acid sequence of: GCGAAGCCGCGAGG (SEQ ID NO: 6), or a sequence with at least 90% sequence identity to SEQ ID NO: 6. In some embodiments, the conditional fluorescent hybridization probe emits fluorescence when hybridized to a nucleic acid molecule comprising a nucleic acid sequence having at least 90%, 91%, 92%, 93%, 94%, 95%, 96%, 97%, 98%, or 99% sequence identity to SEQ ID NO: 6. In some embodiments, the conditional fluorescent hybridization probe emits fluorescence when hybridized to a nucleic acid molecule comprising a nucleic acid sequence having 100% sequence identity to SEQ ID NO: 6.

In some embodiments, the set of detection mixture components comprises Probe 7 ROX and RNase H2.

In some embodiments, the set of detection mixture components further comprises any of the HDA components described above.

In some embodiments, the set of detection mixture components comprises components for HDA and reverse transcription.

In some embodiments, the set of detection mixture components further comprises components for amplicon control, such as the components described above.

In some embodiments, the set of detection mixture components comprises one or more of: Probe 7 ROX, RNase H2, a helicase, an energy source, DNA polymerase, reverse transcriptase, dNTPs, and primers. In some embodiments, the set of detection mixture components comprises Probe 7 ROX, RNase H2, a helicase, an energy source, DNA polymerase, reverse transcriptase, dNTPs, and primers. In some embodiments, the set of components further comprises a single stranded binding protein. In some embodiments, the set of components further comprises a UDG, an enzyme that cleaves DNA at apurinic sites, and dUTP. In some embodiments, the set of components further comprises a Tris buffer, MgSO₄, KCl, NaCl, DTT, Tween-20, sucrose, dextran and/or BSA.

In some embodiments, the primers of a detection mixture comprise one or more primers having hybridization specificity for a single-stranded nucleic acid region comprising a nucleic acid sequence of the target RNA (and/or the corresponding DNA sequence) and a primer having hybridization specificity for a single-stranded nucleic acid region comprising a nucleic acid sequence complementary to the target RNA sequence. In some embodiments, the primers of a detection mixture of the present disclosure comprise the blocked forward primer eF2, the blocked reverse primer R3 and the unblocked reverse primer R12.

In some embodiments, the set of detection mixture components further comprises a DNA restriction enzyme. The DNA restriction enzyme may be used to decrease unwanted exclusive signal in a detection assay of the present disclosure. In some embodiments, the DNA restriction enzyme is included in the assay at a very low concentration relative to typical concentrations used for a restriction enzyme in a standard protocol. In some embodiments, the DNA restriction enzyme is included in the assay at a 100-fold lower concentration as compared to typical concentrations used for a restriction enzyme protocol. For example, a standard restriction enzyme may be used at 10 U per reaction, whereas in the present disclosure, the restriction enzyme may be used at 0.1 U per reaction. In some embodiments, the DNA restriction enzyme is TseI. In some embodiments, TseI is included at 0.1 U per reaction. TseI is active at 65° C. In some embodiments, TseI is active at or near the optimal temperature of a detection assay of the present disclosure. For example, the optimal temperature is the temperature at which amplification occurs. In some embodiments, the optimal temperature of a detection assay of the present disclosure is 64° C. In some embodiments, TseI is included in a detection assay for L. monocytogenes that comprises an amplification step at 64° C. In some embodiments, the detection assay for L. monocytogenes comprises HDA and the set of primers used in the assay comprises an eF2 blocked forward primer, an R3 blocked reverse primer and an R12 unblocked reverse primer. The cDNA of exclusive Listeria species contains a unique TseI recognition and cleavage site not present in L. monocytogenes cDNA. Without wishing to be bound by theory, when exclusive Listeria cDNA is produced by the unblocked reverse primer (R12), TseI binds and cleaves the amplicon at the 3′ end. This destabilizes the binding of the blocked reverse primer (R3) by significantly reducing its melting temperature (Tm) with the cDNA.

In some embodiments, a control RNA molecule is included in an HDA reaction to serve as an internal positive control. In some embodiments, the control RNA molecule is a competitive control, meaning it binds and competes for the L. monocytogenes-specified primers (both reverses and forward) for its amplification. Without wishing to be bound by any theory, inclusion of a competitive control may reduce and/or eliminate the weak amplification of exclusive Listeria RNA.

In some embodiments, the set of detection mixture components further comprises a control RNA and a control probe that is able to detect the control RNA. In some embodiments, the control probe is able to detect amplification products of the control RNA. In some embodiments, amplification products of the control RNA are amplified by one or more of the same primers as those used for reverse transcription and/or HDA in the detection mixture. In some embodiments, the set of detection mixture components further comprises a control reverse primer that can be used for initializing reverse transcription of the control RNA. In some embodiments, one or more bases of the control RNA is fluorinated. In some embodiments, each base of the control RNA is fluorinated. Use of fluorinated bases may reduce or prevent degradation by nucleases. In some embodiments, two types of modifications are used to prevent nuclease degradation: each base is fluorinated and adenine bases with phosphorothioate linkages are placed at the 3′ and 5′ ends of the RNA template to further protect the RNA template. In some embodiments, five adenine bases with phosphorothioate linkages are placed at the 3′ and 5′ ends of the RNA template. In some embodiments, the control RNA is designed to be a competitive or semi-competitive control in an HDA assay of the present disclosure. In some embodiments, amplification products of the control RNA are amplified by the same blocked forward primer as the blocked forward primer used for L. monocytogenes amplification (e.g., eF2 primer in Table 1A). In some embodiments, a separate, unique reverse primer is used for amplification of the control RNA. In some embodiments, the control probe and control reverse primer are designed to ensure no unwanted interactions with the test probe (e.g., Probe 7 ROX). Exemplary sequences of a control RNA, control probe and control reverse primer are provided in Table 1B. In Table 1B, fluorinated bases are annotated as ‘f’ and phosphorothioate linkages as [Ps].

TABLE 1B
Control probe and primer

| SEQ ID | Oligo | | |
|---|---|---|---|
| NO. | Name | Sequence 5′-3′ | Description |
| 7 | LM_CP5- | /HEX/AGA GAG TCT A[rG]T ATC GAC ATG GAT/BHQ-1 | Control |
| | HEX | | Probe |
| 8 | SCR4 | CCGACTTCACCTGCTCGAGTAAAT | Control |
| | | | Reverse |
| | | | Primer |
| 9 | Lm_SC4 | [fA][Ps][fA][Ps][fA][Ps][fA][Ps][fA][Ps][fG][fG][fC][fU][fA][fC] | Fluorinated |
| | | [fA][fC][fA][fC][fG][fU][fG][fC][fU][fA][fC][fA][fA][fU][fG] | RNA control |
| | | [fG][fA][fU][fA][fG][fU][fA][fC][fA][fA][fA][fA][fC][fA][fG] | Template |
| | | [fA][fG][fA][fG][fA][fG][fU][fC][fU][fA][fG][fU][fA][fU][fC] | |
| | | [fG][fA][fC][fA][fU][fG][fG][fA][fU][fU][fA][fU][fU][fU][fA] | |
| | | [fC][fU][fC][fG][fA][fG][fC][fA][fG][fG][fU][fG][fA][fA][fG] | |
| | | [fU][fC][fG][fG][Ps][fA][Ps][fA][Ps][fA][Ps][fA][Ps] | |

In some embodiments, the detection mixture is lyophilized.

In some embodiments, a detection mixture contacted with a composition or mixture comprising a nucleic acid template for the nucleic acid to be detected by components in the detection mixture generates an assay mixture. As used herein, an “assay mixture” refers to a mixture comprising components of a detection mixture and a nucleic acid template for amplification of nucleic acids detected by the probe in the detection mixture. In some embodiments, the nucleic acid template is an RNA (or a region of the RNA) that is reverse transcribed in the assay mixture into a cDNA and the cDNA is amplified and detected by the probe. In some embodiments, an assay mixture is generated when a lysate or an aliquot of a lysate is contacted with a detection mixture. In some embodiments, a method of the present disclosure comprises reverse transcribing a target RNA of a target bacteria in the assay mixture to form target cDNA and amplifying the target cDNA by helicase-dependent amplification (HDA). In some embodiments, a method of the present disclosure also comprises detecting presence or absence of the amplified target cDNA, thereby determining the presence or absence of the target bacteria in the sample.

In some embodiments, an isothermal program is used for detecting presence or absence of target bacteria. In some embodiments, the program for detecting presence or absence of target bacteria comprises: pre-incubation at 37° C. for 20 minutes and amplification at 64° C. for 100 minutes with fluorescence measurement every 30 seconds. In some embodiments, the program for detecting presence or absence of target bacteria is processed using a qPCR instrument. In some embodiments, the program for detecting presence or absence of target bacteria is processed using a CFX96 qPCR or Optigene instrument.

### L. Monocytogenes Detection Assays

The present disclosure provides methods for detection of L. monocytogenes.

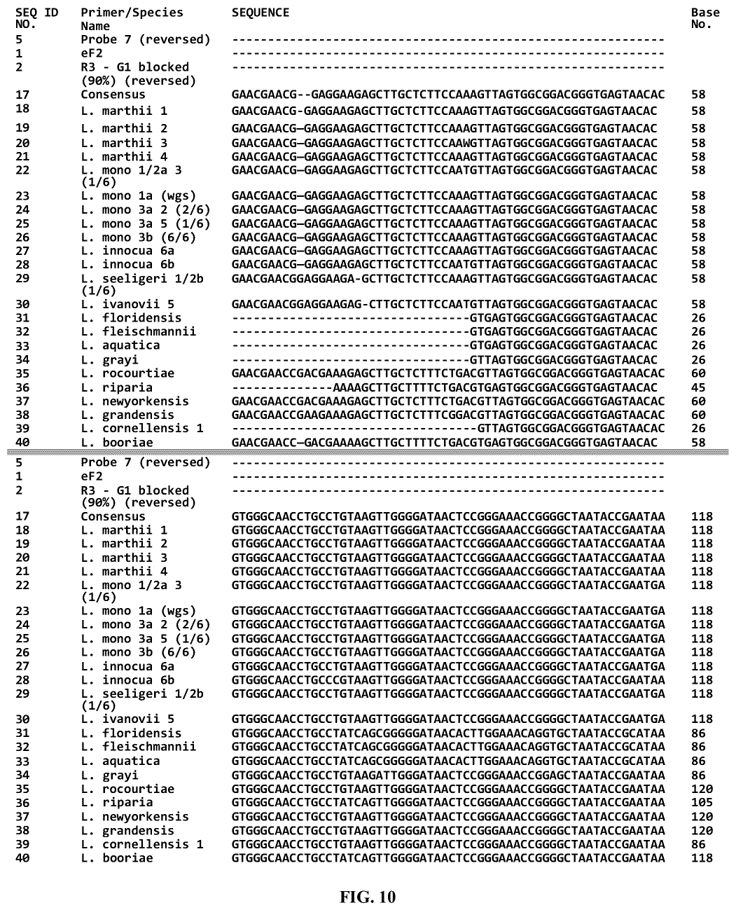

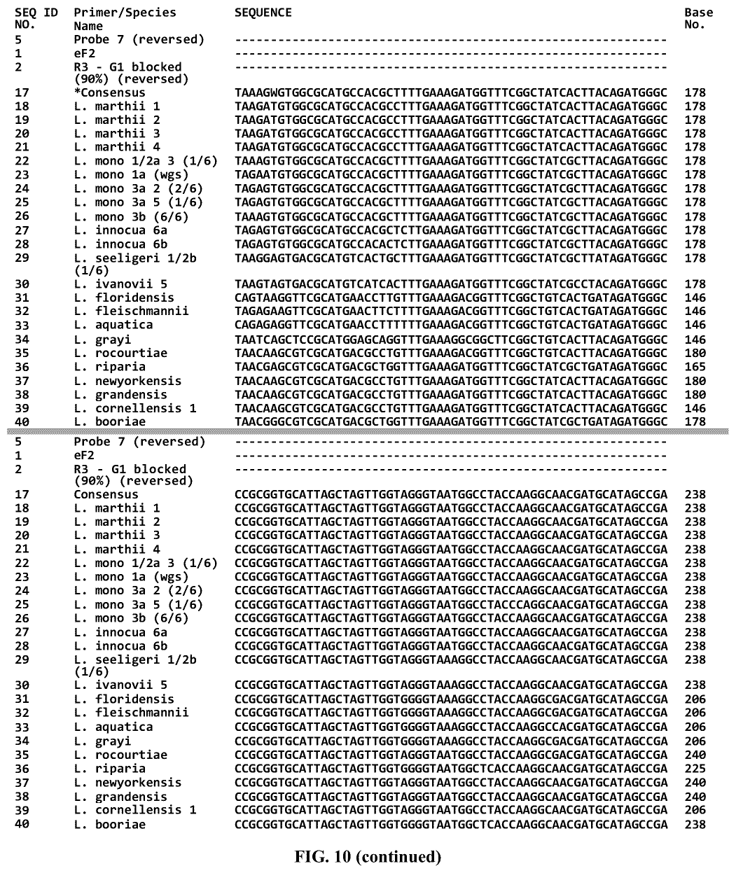

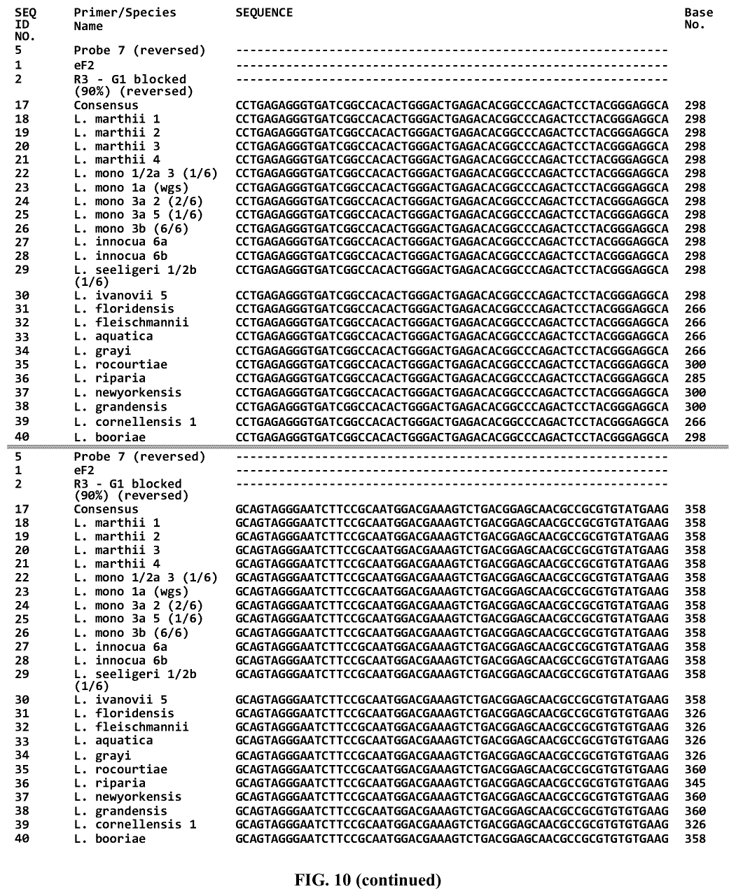

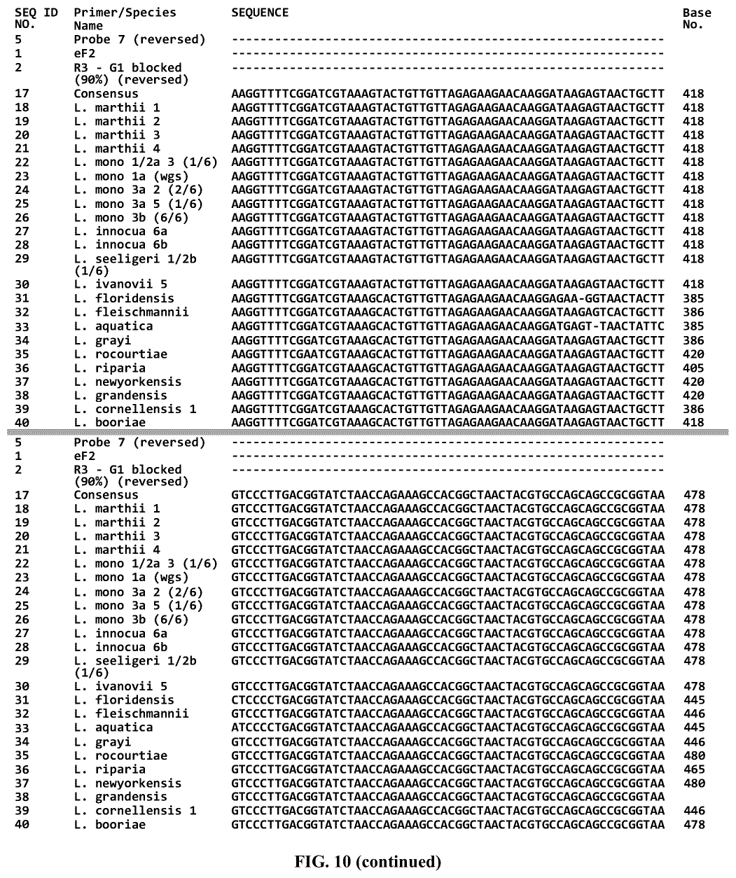

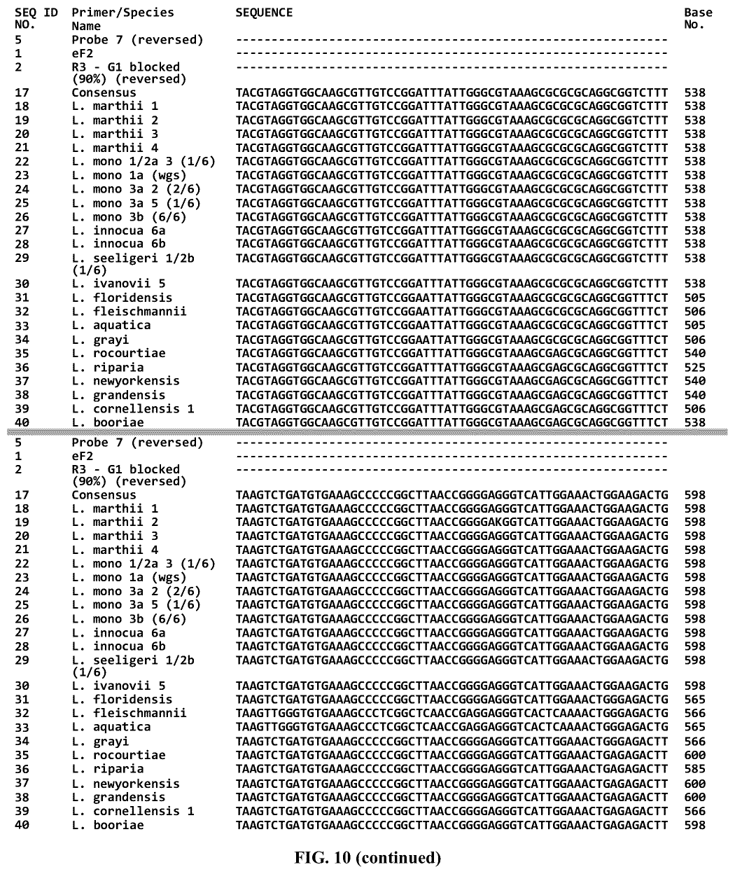

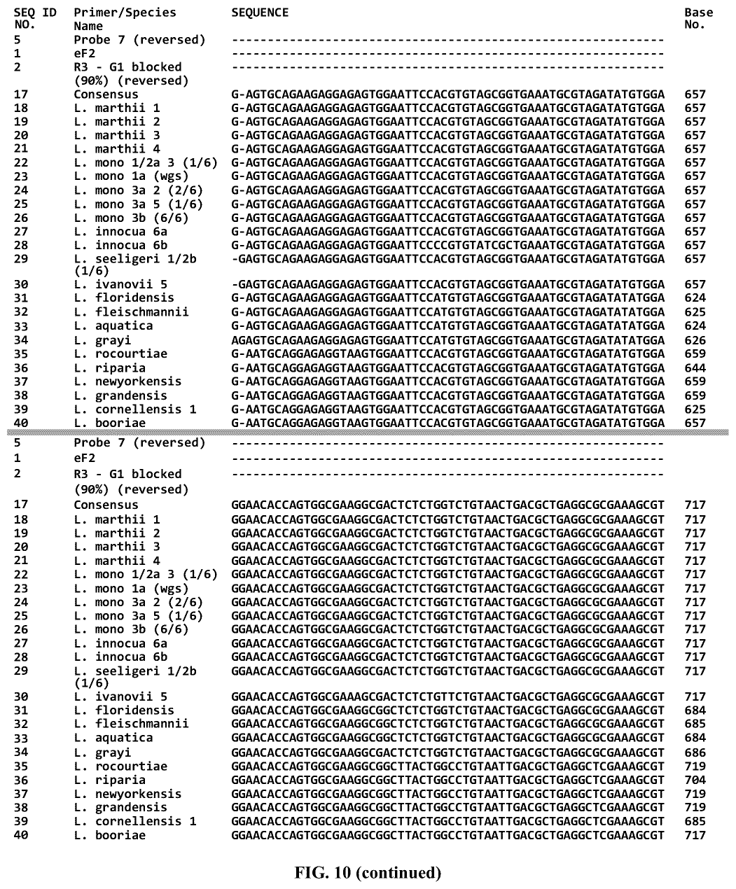

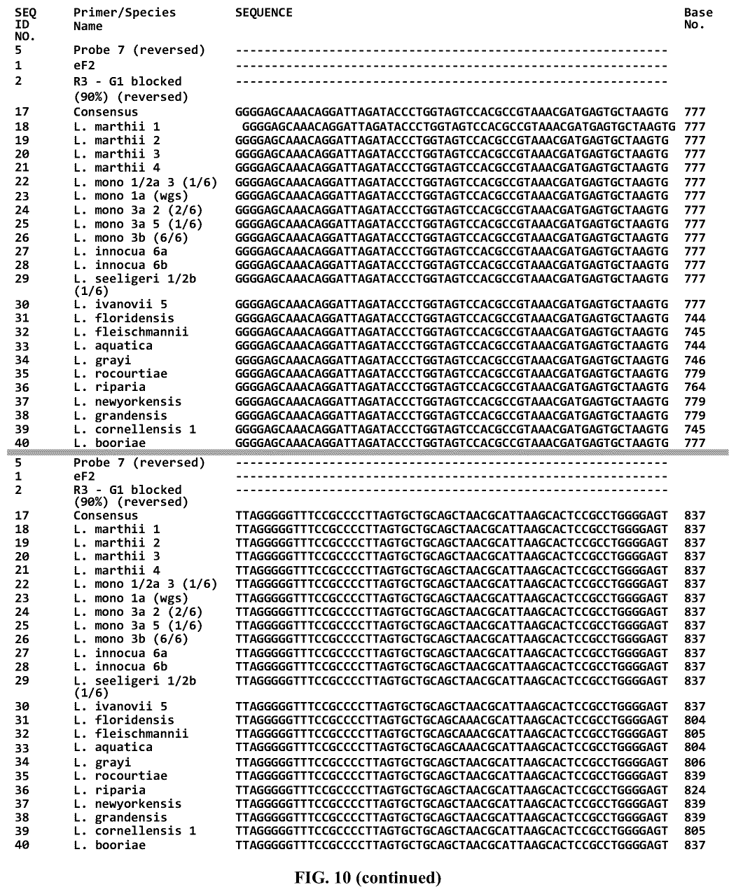

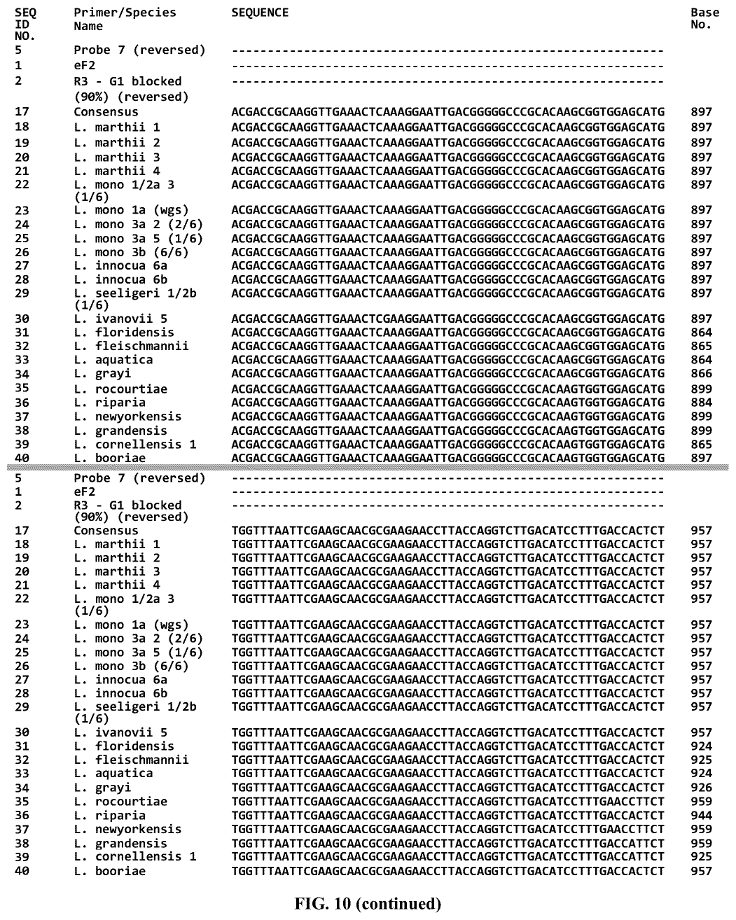

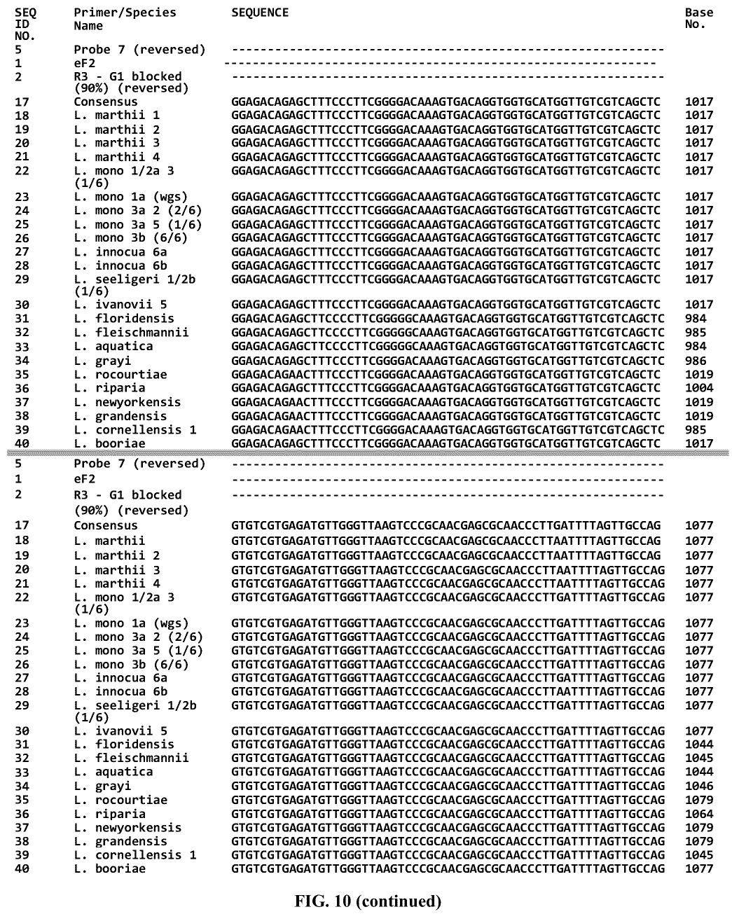

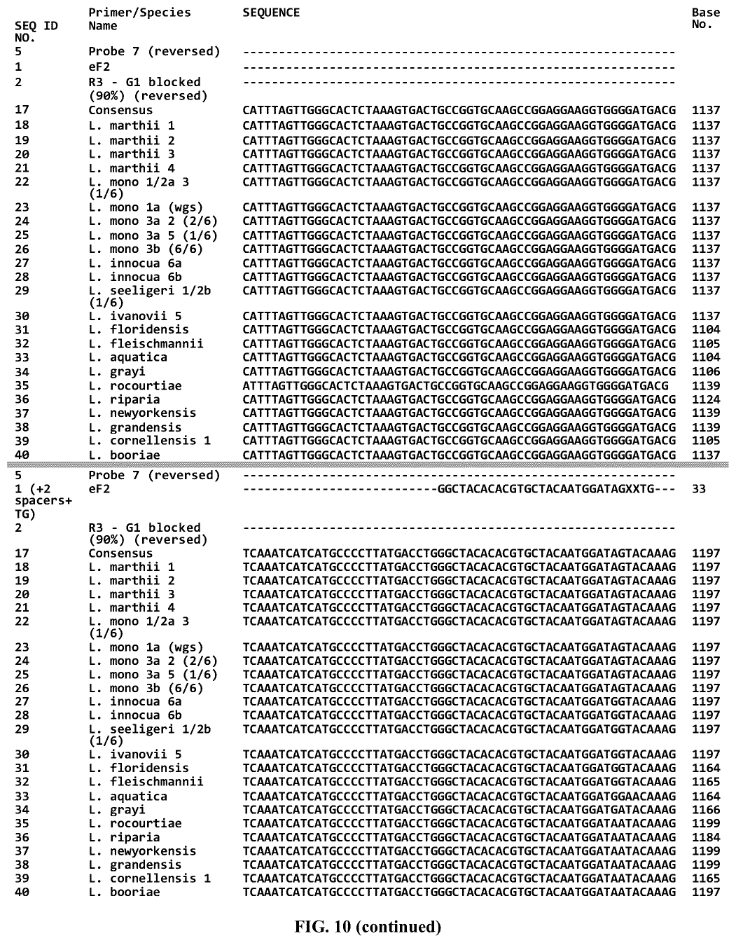

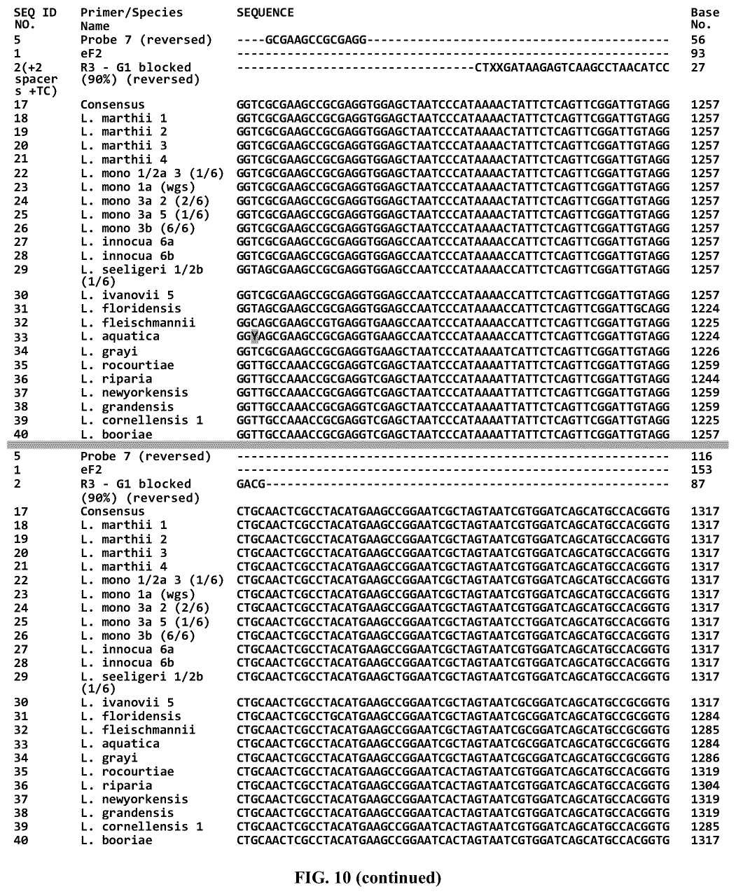

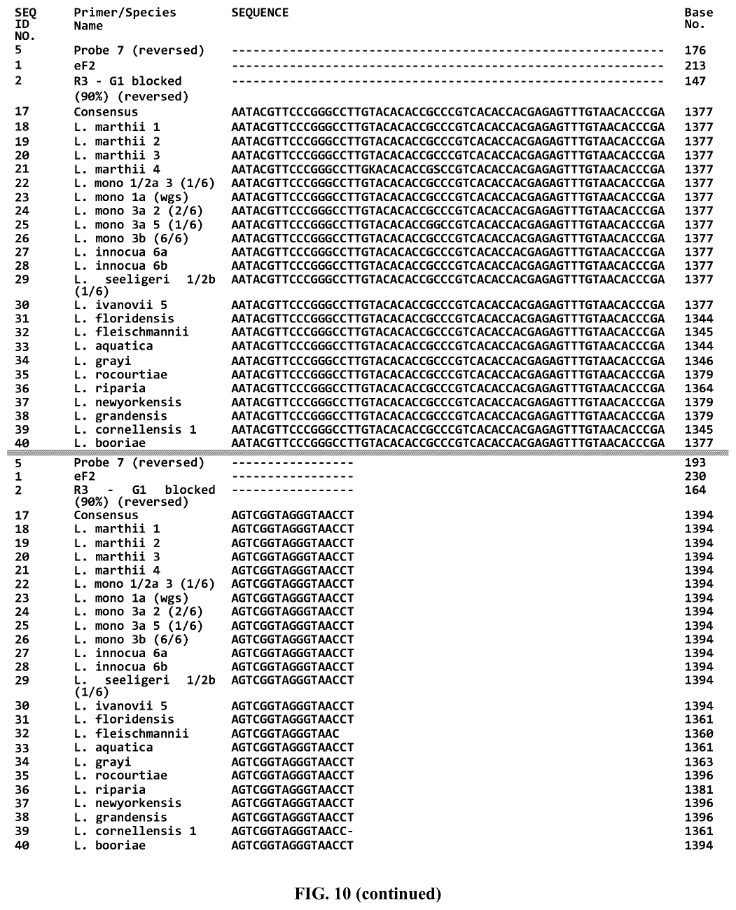

In various aspects, provided herein are assays developed useful in applications such as environmental sample diagnostics with high sensitivity to L. monocytogenes. In various embodiments, the provided assays target the 16S ribosomal RNA (rRNA) of L. monocytogenes. In some embodiments, the provided assays target all serotypes of L. monocytogenes (see FIG. 10; SEQ ID NOs: 18-40). The Listeria species excluded from the provided assays are listed in Table 1C and also FIG. 10. Due to identical sequences within the target region, the provided assays are unable to differentiate Listeria marthii and Listeria cossartiae from L. monocytogenes. All three species are detected.

TABLE 1C
Inclusive and exclusive species

| Assay | | |
|---|---|---|
| Design | Genus | Species |
| Inclusive | Listeria | monocytogenes |
| Exclusive | Listeria | innocua |
| Exclusive | Listeria | seeligeri |
| Exclusive | Listeria | grayi |
| Exclusive | Listeria | fleishchmannii |
| Exclusive | Listeria | riparia |
| Exclusive | Listeria | grandensis |
| Exclusive | Listeria | cornellensis |
| Exclusive | Listeria | ivanovii |
| Exclusive | Listeria | aquatica |
| Exclusive | Listeria | floridensis |
| Exclusive | Listeria | rocourtiae |
| Exclusive | Listeria | newyorkensis |
| Exclusive | Listeria | booriae |

In various embodiments, the present disclosure provides methods of detecting L. monocytogenes from environmental samples. In some embodiments, the present disclosure provides methods of detecting L. monocytogenes from an environmental sample without the need for enrichment or a prolonged incubation period. The provided methods for L. monocytogenes detection combine various individual methods described above (e.g., lysis, HDA, amplicon control and/or detection/quantification of target nucleic acids) for the purposes of detecting and/or quantifying L. monocytogenes from a sample.

In some embodiments, methods for detecting L. monocytogenes provided herein combine reverse transcription of a target RNA with amplification of the resulting cDNA. In various embodiments, reverse transcription and DNA amplification occur at the same time and in the same reaction vessel. Descriptions of concurrent reverse transcription and DNA amplification are provided above, for example, in the “Helicase-dependent amplification” section.

In addition to the above-described concurrent reverse transcription and DNA amplification, the provided methods for detection of L. monocytogenes also combine various other methods described throughout the present disclosure to detect and/or quantify L. monocytogenes in a sample.

A non-limiting example of the combination of methods from the present disclosure that can be used for detection of L. monocytogenes comprise: (i) combined reverse transcription and DNA amplification via thermostable helicase-dependent amplification (tHDA) and (ii) detection of amplified nucleic acids.

Another non-limiting example of the combination of methods from the present disclosure that can be used for detection of L. monocytogenes comprise: (i) bacterial cell lysis; (ii) combined reverse transcription and DNA amplification via thermostable helicase-dependent amplification (tHDA); and (iii) detection of amplified nucleic acids.

Another non-limiting example of the combination of methods from the present disclosure that can be used for detection of L. monocytogenes comprise: (i) bacterial cell lysis; (ii) amplicon control to prevent prior positive reactions from contaminating and triggering false positives on subsequent negative reactions; (iii) combined reverse transcription and DNA amplification via thermostable helicase-dependent amplification (tHDA); and (iv) detection of amplified nucleic acids.

In various embodiments, detection of amplified nucleic acids comprises use of at least one fluorescent hybridization probe that recognizes the amplified nucleic acids.

In various embodiments of the present disclosure, the combination of methods for detection of L. monocytogenes in a provided sample are combined in a series of steps that occur in the following sequence: (i) cell lysis; (ii) amplicon control; and (iii) reverse transcription, DNA amplification, and detection of amplified target. In various embodiments, nucleic acid amplification occurs at a single temperature (usually between about 55-68° C.). In some embodiments, nucleic acid amplification occurs at or above 45° C. and at or below 70° C. In some embodiments, nucleic acid amplification occurs at 64° C.

In some embodiments, the combination of methods for detection of L. monocytogenes comprises use of a lysis mixture and/or a detection mixture. For example, any of the above-described lysis and detection mixtures may be used for detection of L. monocytogenes according to the present disclosure.

As a non-limiting example, the lysis mixture comprises: (i) lysozyme; (ii) mutanolysin; (iii) proteinase K; (iv) achromopeptidase; (v) Chelex®-100; (vi) Tris HCl, pH 8.8; (vii) EGTA; (viii) dextran; and (ix) sucrose. In some embodiments, the lysis mixture components are lyophilized as one or more lyophilized pellets. In some embodiments, the set of lysis mixture components comprises the components listed in Table 4 and the concentration of each of the listed components after resuspension of the lyophilized lysis mixture components is the concentration listed in Table 4.

As a non-limiting example, the detection mixture comprises: (i) a Probe? ROX; (ii) RNase H2; (iii) a helicase; (iv) an energy source in the form of dATP; (v) DNA polymerase; (vi) reverse transcriptase; (vii) dNTPs; (viii) forward and reverse primers; (ix) Tris-HCl pH 8.8; (x) KCl; (xi) NaCl; and (xii) magnesium sulfate. In some embodiments, the detection mixture further comprises at least one single stranded binding protein (SSB). In some embodiments, the detection mixture further comprises: (i) UDG, (ii) Endonuclease VIII, and (iii) dUTP. In some embodiments, the detection mixture further comprises: (i) DTT, (ii) Tween-20, (iii) sucrose, (iv) dextran, and/or (v) BSA. In some embodiments, the detection mixture further comprises a control RNA, a control reverse primer, and a control probe that is able to detect the control RNA. In some embodiments, the detection mixture comprises one or more of the components listed in Table 4. In some embodiments, one or more components of the detection mixture are lyophilized together as one lyophilized pellet. In some embodiments, one or more components of the detection mixture are lyophilized separately as more than one lyophilized pellet.

In some embodiments, the primers of a detection mixture comprise one or more primers having hybridization specificity for a single-stranded nucleic acid region comprising a nucleic acid sequence of the target RNA (and/or the corresponding DNA sequence) and a primer having hybridization specificity for a single-stranded nucleic acid region comprising a nucleic acid sequence complementary to the target RNA sequence.

In some embodiments, the detection mixture is lyophilized. In some embodiments, one or more lyophilized pellets, each comprising one or more components of a detection mixture are resuspended. For example, the one or more lyophilized pellets may be resuspended with a liquid composition. In some embodiments, the one or more lyophilized pellets may be resuspended with a sample or solution comprising lysed target bacteria cells (e.g., a lysate or an aliquot thereof). In some embodiments, the concentration of one or more of the components of the detection mixture after resuspension is in the range of concentrations listed in Table 4. In some embodiments, the set of detection mixture components comprises the components listed in Table 4 and the concentration of each of the listed components after resuspension of the lyophilized detection mixture components is in the range specified in Table 4. In some embodiments, the set of detection mixture components comprises one or more of the components listed in Table 5 and the concentration of each component after resuspension of the lyophilized detection mixture components is the concentration listed in Table 5. In some embodiments, the set of detection mixture components comprises the components listed in Table 5 and the concentration of each of the listed components after resuspension of the lyophilized detection mixture components is the concentration listed in Table 5.

TABLE 4
Concentration ranges for detection mixture components

| | Concentration | | | |
|---|---|---|---|---|
| | Range (minimum and | | | |
| Component | maximum values shown) | | | |
| mH20 | | | | |
| Forward Primer: eF2 | 60 | nM | 100 | nM |
| Reverse Primer: R3 | 60 | nM | 100 | nM |
| Reverse Primer: R12 | 5 | nM | 80 | nM |
| Control Reverse Primer SCR4 | 5 | nM | 80 | nM |
| Probe: Pr7-ROX | 40 | nM | 100 | nM |
| Control Probe: LM CP5 HEX | 40 | nM | 100 | nM |
| Control Template: SC4 | 100 | copies/μL | 10⁵ | copies/μL |
| KCl | 8 | mM | 12 | mM |
| Tris-HCl | 15 | mM | 65 | mM |
| MgSO₄ | 8 | mM | 12 | mM |
| NaCl | 25 | mM | 50 | mM |
| dATP | 5.5 | mM | 7.5 | mM |
| dGTP | 0.4 | mM | 1 | mM |
| dCTP | 0.4 | mM | 1 | mM |
| dTTP | 0 | mM | 1 | mM |
| dUTP | 0 | mM | 1 | mM |
| Tsel | 0.001 | Units/μL | 0.2 | Units/μL |
| Antarctic Thermolabile UDG | 0 | Units/μL | 0.2 | Units/μL |
| Tte-UvrD Helicase | 10 | ng/μL | 20 | ng/μL |
| Sso SSB | 2 | ng/μL | 20 | ng/μL |
| Gst Polymerase | 1.8 | Units/μL | 5 | Units/μL |
| Roche RT | 0.3 | U/μL | 0.8 | U/μL |
| RNAseHII | 1 | ng/μL | 20 | ng/μL |
| Endonuclease VIII | 0 | Units/μL | 0.6 | Units/μL |
| DTT | 1.5 | mM | 3 | mM |
| Sucrose | 1% | (m/v) | 6% | (m/v) |
| Dextran | 1% | (m/v) | 5% | (m/v) |
| BSA | 0 | mg/mL | 1 | mg/mL |
| Tween-20 | 0% | (v/v) | 0.2% | (v/v) |

TABLE 5
Detection mixture components and concentrations

| Component | Final Concentration | | Per Reaction | |
|---|---|---|---|---|
| mH20 | | | Add to final | |
| | | | volume of 50 μL | |
| Forward Primer: eF2 | 0.08 | μM | | |
| Reverse Primer: R3 | 0.064 | μM | | |
| Reverse Primer: R12 | 0.016 | μM | | |
| Control Reverse Primer SCR4 | 0.010 | μM | | |
| Probe: Pr7-ROX | 0.08 | μM | | |
| Control Probe: LM CP5 HEX | 0.08 | μM | | |
| Control Template: SC4 | 100 | copies/μL | 5000 | |
| KCl | 10 | mM | | |
| Tris-HCl | 20 | mM | | |
| MgSO₄ | 10 | mM | | |
| NaCl | 30 | mM | | |
| dATP | 6.5 | mM | | |
| dGTP | 0.5 | mM | | |
| dCTP | 0.5 | mM | | |
| dTTP | 0.25 | mM | | |
| dUTP | 0.25 | mM | | |
| Tsel | 0.002 | U/μl | | |
| Antarctic Thermolabile UDG | 0.02 | U/μl | 1 | U |
| Tte-UvrD Helicase | 10 | ng/μl | 500 | ng |
| Sso SSB | 3 | ng/μl | 150 | ng |
| Gst Polymerase | 3 | U/μl | 150 | ng |
| Roche RT | 0.4 | U/μl | 20 | U |
| RNAse HII | 5 | ng/μl | 800 | ng |
| Endonuclease VIII | 0.4 | U/μl | 20 | U |

| DTT | 2 | mM |
|---|---|---|
| Sucrose | 21% | |
| Dextran | 2.5% | |
| BSA | 0.5 | mg/mL |
| Tween-20 | 0.14% | (v/v) |

In some embodiments, methods for detecting L. monocytogenes provided herein comprise the use of components and concentrations provided in Table 4 or Table 5 for reverse transcription of a target RNA with amplification of the resulting cDNA. In some embodiments, an isothermal program is used for detecting L. monocytogenes. In some embodiments, the program for detecting L. monocytogenes comprises: pre-incubation at 37° C. for 20 minutes and amplification at 64° C. for 100 minutes with fluorescence measurement every 30 seconds. In some embodiments, the program for detecting L. monocytogenes is processed using a qPCR instrument. In some embodiments, the program for detecting L. monocytogenes is processed using a CFX96 qPCR or Optigene instrument.

In some embodiments, the limit of detection of a method described herein is 100 fg of L. monocytogenes RNA, or the equivalent of RNA of 1 CFU.

In some embodiments, methods for detecting L. monocytogenes provided herein comprise determining the presence or absence of L. monocytogenes in an environmental sample. In some embodiments, the environmental sample is from an environment comprising a low concentration of L. monocytogenes and the methods described herein are of sufficient sensitivity to detect the presence of L. monocytogenes in the environmental sample. In some embodiments, methods described herein are of sufficient sensitivity to detect presence of L. monocytogenes from a sample having as little as 30-60 CFU of L. monocytogenes. In some embodiments, methods described herein are of sufficient sensitivity to detect presence of L. monocytogenes from a sample having as little as 5-10 CFU of L. monocytogenes. In some embodiments, methods described herein are of sufficient sensitivity to detect presence of L. monocytogenes from a sample having as little as about 1 CFU of L. monocytogenes. In some embodiments, the sample further comprises bacteria that is not L. monocytogenes. In some embodiments, the environmental sample is from a solid surface comprising a low concentration of L. monocytogenes and the methods described herein are of sufficient sensitivity to detect the presence of L. monocytogenes in the environmental sample. In some embodiments, methods described herein are of sufficient sensitivity to detect presence of L. monocytogenes in an environmental sample from a solid surface that comprises from about 5 to about 200 CFU of L. monocytogenes per 1 square inch of the solid surface. In some embodiments, the solid surface comprises from about 5 to about 100 CFU of L. monocytogenes per 1 square inch of the solid surface. In some embodiments, the solid surface comprises from about 5 to about 50 CFU of L. monocytogenes per 1 square inch of the solid surface. In some embodiments, the solid surface further comprises microflora that is not L. monocytogenes. In various embodiments wherein the sample or environment from where the sample is collected comprises L. monocytogenes and microflora that is not L. monocytogenes, the methods described herein are of sufficient sensitivity and specificity to detect presence of L. monocytogenes.

Compositions and Kits

The present disclosure also provides compositions and kits that may be used in the methods described herein.

In one aspect, the disclosure provides a lyophilized lysis composition comprising one or more components of a lysis mixture. In some embodiments, the lyophilized lysis composition comprises (i) at least one of lysozyme and mutanolysin; (ii) at least one of proteinase K and achromopeptidase; and (iii) EGTA. In some embodiments, the concentration of each component of the lyophilized lysis composition after resuspension (for example, resuspension with a liquid composition, e.g., resuspension with a sample or solution comprising target bacteria cells or resuspension with a pre-treated sample or aliquot thereof) is the concentration listed in Table 4.

In some embodiments, the present disclosure provides a lyophilized composition comprising (i) at least one of lysozyme and mutanolysin; (ii) at least one of proteinase K and achromopeptidase; and (iii) EGTA, wherein, upon resuspension of the lyophilized composition, the concentration of lysozyme ranges from 0-1 mg/mL, the concentration of mutanolysin ranges from 0-30 Units/mL, the concentration of proteinase K ranges from 0-1 mg/mL, the concentration of achromopeptidase ranges from 0-150 Units/mL, and the concentration of EGTA ranges from 2-5 mM. In some embodiments, upon resuspension of the lyophilized composition, the concentration of lysozyme is 0.8 mg/mL, the concentration of mutanolysin is 20 Units/mL, the concentration of proteinase K is 0.8 mg/mL, the concentration of achromopeptidase is 85.6 Units/mL, and the concentration of EGTA is 2.6 mM.

In one aspect, the disclosure provides a lyophilized detection mixture composition comprising one or more components of a detection mixture. The one or more components may be selected from components listed in Table 5.

In some embodiments, the disclosure provides a lyophilized composition comprising three primers: eF2 primer, R3 primer and R12 primer, as described in Table 1A. In some embodiments, the disclosure provides a lyophilized composition comprising four primers: eF2 primer, R3 primer and R12 primer, as described in Table 1A and SCR4 primer as described in Table 1B.

Aspects of the disclosure also include kits, the kits comprising components and/or compositions used in the methods described herein.

In some embodiments, a kit of the present disclosure may be for use in a method of determining the presence or absence of target bacteria in an environmental sample.

In some embodiments, a kit may comprise one or more of: a collection device, lysis mixture or components thereof, detection mixture or components thereof, equipment (e.g., optical reader), reagents (primers, probes, dNTPs, enzymes, etc.), and instructions for use.

In some embodiments, a kit comprises a lysis mixture and a detection mixture. In some embodiments, one or more of the lysis mixture and the detection mixture are lyophilized. In some embodiments, one or more components of the lysis mixture and the detection mixture are lyophilized.

In some embodiments, a kit comprises two primers. In some embodiments, a kit comprises two blocked primers. In some embodiments, a kit comprises a first blocked primer comprising an oligonucleotide comprising the sequence GGC TAC ACA CGT GCT ACA ATG GAT (SEQ ID NO: 10) and a second blocked primer comprising an oligonucleotide comprising the sequence GCA GCC TAC AAT CCG AAC TGA GAA T (SEQ ID NO: 11).

In some embodiments, a kit further comprises an unblocked primer that comprises or consists of the sequence: CGA GTT GCA GCC TAC AAT CCG AAC TGA G (SEQ ID NO: 3). In some embodiments, the kit further comprises at least one probe for detecting a nucleic acid molecule of interest. In some embodiments, the kit comprises at least one probe that comprises the nucleic acid sequence of: CCT CGC GGrC TTC GC (SEQ ID NO: 5), or a sequence with at least 90% sequence identity thereto, wherein the probe comprises DNA bases and a single RNA base, and wherein the “r” denotes that the base is RNA. In some embodiments, a kit comprises an eF2 primer and an R3 primer, as shown in Table 1A. In some embodiments, a kit further comprises an R3 primer, as shown in Table 1A. In some embodiments, a kit further comprises Probe 7 ROX, as shown in Table 1A. In some embodiments, one or more primers and/or the probe are lyophilized.

In some embodiments, a kit comprises a control RNA template and a control probe. In some embodiments, the control RNA template is Lm_SC4 and the control probe is LM_CP5-HEX, as shown in Table 1B. In some embodiments, a kit further comprises a control reverse primer. In some embodiments, the control reverse primer comprises the sequence: CCGACTTCACCTGCTCGAGTAAAT (SEQ ID NO: 8). In some embodiments, a kit comprises one or more of the primers shown in Table 1A and one or more of the primer, probe and control RNA template shown in Table 1B.

### EXAMPLES

The following examples are offered by way of illustration, not by way of limitation.

### Example 1: Design of Blocked Primers for Amplification of L. monocytogenes Target Nucleic Acids

The present example demonstrates amplification of nucleic acids comprising a target sequence of L. monocytogenes using blocked primers.

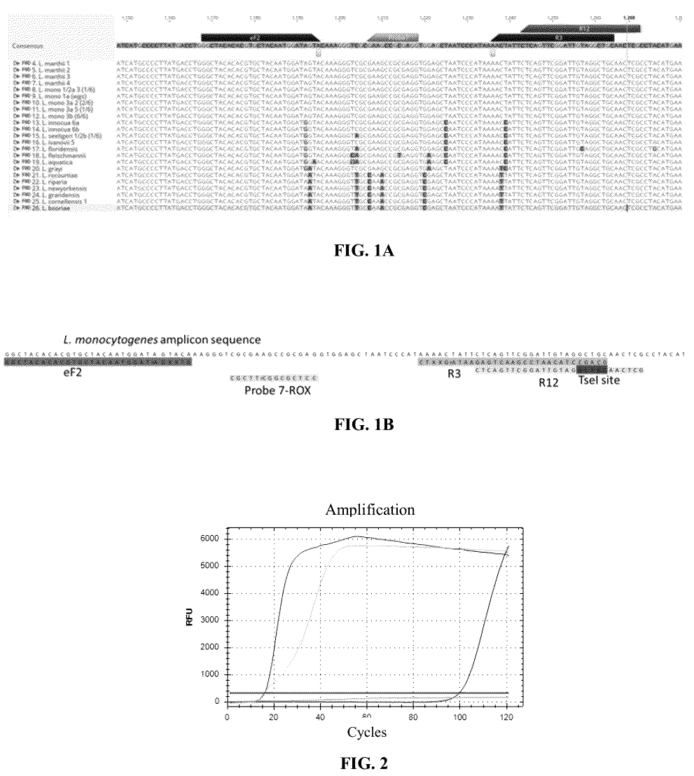

The forward blocked rhPCR GEN2 primer, eF2, and the reverse blocked rhPCR GEN2 primer, R3, were designed to target single nucleotide polymorphisms (SNPs) specific to L. monocytogenes and inhibit amplification of Listeria innocua, Listeria ivanovii and Listeria welshimeri. These primers, herein also referred to as “RNase H-dependent PCR (rhPCR) primers,” can be activated for polymerization upon cleaving of the 3′ blocking moiety by the enzyme RNaseHII. The GEN2 rhPCR primers are designed with a rDXXDM blocking group, where ‘f is a ribobase complementary to a L. monocytogenes specific SNP, ‘D’ is a DNA base followed by two ‘X’ C3 spacers or phosphoramidite, an additional DNA base and a ‘M’ or 3’ intentional mismatch to increase specificity of the RNaseHII cleavage. Mismatch bases are underlined in Table 1A. A gene block fragment dsDNA template was purchased from Integrated DNA Technologies, Inc. (Coralville, IA) for blocked primer testing (sequences listed in Table 6 and FIG. 1B).

To test the blocked primers the following reagents were set up in 50 μL reactions. 20 mM Tris pH 8.8, 10 mM MgSO₄, 10 mM KCL, 30 mM NaCl, 6.5 mM dATP, 0.5 mM dGTP, 0.5 mM dCTP, 0.2 mM dTTP, 0.2 mM dUTP, 1 U Antarctic Thermolabile UDG, 500 ng Tte-UvrD Helicase, 150 ng Sso SSB, 150 ng Gst Polymerase, 20 U Roche RT, 800 ng RNAseHII, 20 U Endonuclease VIII. 80 nM eF2, 80 nM R3, 0.5× Evagreen dye, and either 1 ng of DNA, RNA or a no template control. Positive control reaction had the same master mix with 80 nM HDA_F1, 80 nM HDA_R1, 1 ng positive control template, HDA GB.

The Cfx program was 37° C. for 20 minutes followed by a 64° C. step for 120 minutes with FAM fluorescent reads every 30 seconds to monitor intercalation of Evagreen dye. A Melt curve was then done by raising the temperature every 30 secs from 64° C. to 95° C. to monitor denaturation of any amplicon products.

Amplification was observed with the addition of an intercalating dye, EvaGreen (FIG. 2). Purified L. monocytogenes RNA was also tested with two blocked primers, and resulted in no amplification, indicating reverse transcriptase requires a standard reverse primer.

As demonstrated by the present example, the blocked forward and blocked reverse primers described herein enable amplification of DNA molecules comprising a targeted L. monocytogenes sequence.

### Example 2: Combination of Blocked and Unblocked Primers for Amplification of L. monocytogenes from Target RNA

The present example demonstrates amplification of nucleic acids comprising a target sequence of L. monocytogenes from an RNA starting material. This example also describes approaches for reducing amplification of unwanted (exclusive) bacterial nucleic acids.

As described in Example 1 above, a blocked reverse primer was designed for amplification of L. monocytogenes target nucleic acids. To amplify nucleic acids from an RNA starting material, the present example uses a standard (unblocked) reverse primer with a reverse transcriptase to synthesize cDNA followed by use of the above-described blocked reverse primer to amplify the cDNA.

A standard reverse primer, R12, was selected for the reverse transcription of L. monocytogenes 16S rRNA target and polymerization of cDNA. This standard reverse primer does not increase specificity to the assay and will amplify all exclusive targets. To reduce exclusive signal, a ratio of blocked to unblocked primer was optimized.

A combination of blocked and unblocked primers were used in a HDA assay. Test samples included 1 ng of purified L. monocytogenes (inclusive bacteria), 1 ng of purified L. innocua RNA (exclusive bacteria), and a no template control. To further decrease unwanted exclusive signal, the use of a DNA restriction enzyme, TseI was introduced to the assay at a very low concentration. Standard restriction enzymes are often used at 10 U per reaction while TseI is at 0.1 U per reaction. TseI is active at 65° C., near the optimal assay temperature, 64° C. TseI has a restriction site in the reverse primer, R12.

To test the incorporation of an unblocked reverse primer, R12, the following reagents were set up in 50 uL reactions. 20 mM Tris pH 8.8, 10 mM MgSO₄, 10 mM KCL, 30 mM NaCl, 6.5 mM dATP, 0.5 mM dGTP, 0.5 mM dCTP, 0.2 mM dTTP, 0.2 mM dUTP, 1 U Antarctic Thermolabile UDG, 500 ng Tte-UvrD Helicase, 150 ng Sso SSB, 150 ng Gst Polymerase, 20 U Roche RT, 800 ng RNAseHII, 20 U Endonuclease VIII. 80 nM eF2, 64 nM R3, 12 nM R12, 0.5× Evagreen dye, and either 1 ng of purified L. monocytogenes RNA, 1 ng of L. innocua RNA or a no template control.

The Cfx program was 37° C. for 20 minutes followed by a 64° C. step for 60 minutes with FAM fluorescent reads every 30 seconds to monitor intercalation of Evagreen dye. A Melt curve was then done by raising the temperature every 30 secs from 64° C. to 95° C. to monitor denaturation of any amplicon products.

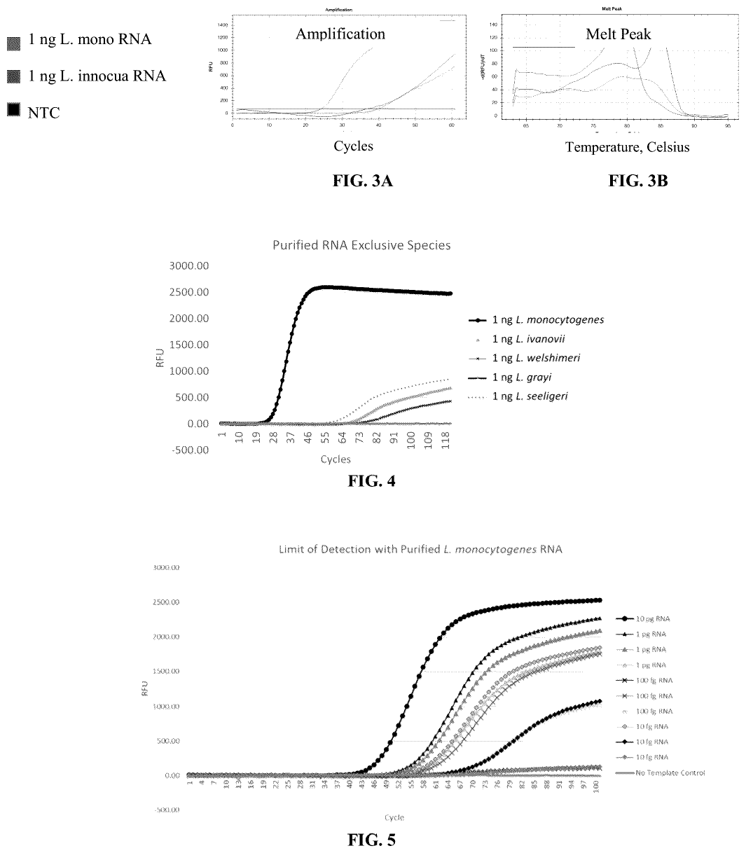

The HDA assay results indicated that a combination of 64 nM of R3 (blocked primer) and 16 nM of R12 (standard reverse primer) allowed for strong L. monocytogenes signal with minimal signal from L. innocua (FIGS. 3A and B).

As demonstrated by the present example, a combination of blocked and unblocked reverse primers can be used to amplify target L. monocytogenes nucleic acids from an RNA starting material and to exclude unwanted amplification of nucleic acids from exclusive Listeria. Also as shown here, the blocked and unblocked reverse primers can be combined for use in a HDA assay for amplification of L. monocytogenes nucleic acids.

### Example 3: Probe Design for Increased Selectivity of the Assay

The present example demonstrates that a detection probe can be designed to increase selectivity of a L. monocytogenes detection assay.

A probe was designed to include a ribobase to bind a L. monocytogenes-specific SNP. L. monocytogenes-specific SNP were determined from the alignment in FIGS. 1A and 10. Primers and probe were tested against total RNA purified from Listeria organisms at 1 ng/reaction.

To test selectivity of the assay, the following reagents were set up in 50 μL reactions. 20 mM Tris pH 8.8, 10 mM MgSO₄, 10 mM KCL, 30 mM NaCl, 6.5 mM dATP, 0.5 mM dGTP, 0.5 mM dCTP, 0.2 mM dTTP, 0.2 mM dUTP, 1 U Antarctic Thermolabile UDG, 500 ng Tte-UvrD Helicase, 150 ng Sso SSB, 150 ng Gst Polymerase, 20 U Roche RT, 800 ng RNAseHII, 20 U Endonuclease VIII, 0.1 U TseI. 80 nM eF2, 64 nM R3, 12 nM R12, 80 nM Probe7 ROX, 2 mM DTT, 2% Sucrose, 2.5% Dextran, 25 mg BSA, 0.14% Tween20 and either 1 ng of purified RNA.

The Cfx program was 37° C. for 20 minutes followed by a 64° C. step for 120 minutes with ROX fluorescent reads every 30 seconds to monitor test probe cleavage.

In contrast to several exclusive Listeria species (e.g., Listeria riparia, Listeria grayi and Listeria booriae), amplification of L. monocytogenes was robust and early (FIG. 4).

As demonstrated by the present example, a detection probe can be designed to increase selectivity of a L. monocytogenes detection assay and to reduce unwanted signal from exclusive Listeria species.

### Example 4: Limit of Detection Analysis for L. monocytogenes Detection Assay

Primers and probe designed for L. monocytogenes were tested against serially diluted purified RNA from L. monocytogenes.

To test the limit of detection of the assay, the following reagents were set up in 50 μL reactions. 20 mM Tris pH 8.8, 10 mM MgSO₄, 10 mM KCL, 30 mM NaCl, 6.5 mM dATP, 0.5 mM dGTP, 0.5 mM dCTP, 0.2 mM dTTP, 0.2 mM dUTP, 1 U Antarctic Thermolabile UDG, 500 ng Tte-UvrD Helicase, 150 ng Sso SSB, 150 ng Gst Polymerase, 20 U Roche RT, 800 ng RNAseHII, 20 U Endonuclease VIII, 0.1 U TseI. 80 nM eF2, 64 nM R3, 12 nM R12, 80 nM Probe? ROX, 2 mM DTT, 2% Sucrose, 2.5% Dextran, 25 mg BSA, 0.14% Tween20 and serially diluted purified RNA.

The Cfx program was 37° C. for 20 minutes followed by a 64° C. step for 120 minutes with ROX fluorescent reads every 30 seconds to monitor test probe cleavage.

Signal was observed past 100 femtograms of RNA (FIG. 5), which is roughly equivalent to 1 colony forming unit (CFU) per reaction.

### Example 5: Inclusivity Analysis for L. monocytogenes Detection Assay

Two serotypes of L. monocytogenes were tested for inclusivity. The L. monocytogenes target sequence is shared with all of the L. monocytogenes serotypes. It is expected that all serotypes will be detected by the assay. In the present example, two serotypes of L. monocytogenes, 1/2a and 4c, were tested.

Overnight cultures of the Listeria to be tested were grown in LESS Plus growth media at 37° C. 100 μL of the serially diluted culture in Buffered Peptone Buffer (BPB) was added to chelex and 400 μL of lysis enzymes. The cells and lysis mixture was vortexed at 1000 rpm for 10 seconds and incubated at 37° C. for 20 minutes. The lysis mixture was vortexed again at 1000 rpm for 10 seconds and incubated at 95° C. for 8 minutes. 5 μL of lysed cells was added to the assay reaction.

To test inclusivity of the assay, the following reagents were set up in lyophilized reactions. 20 mM Tris pH 8.8, 10 mM MgSO₄, 10 mM KCL, 30 mM NaCl, 6.5 mM dATP, 0.5 mM dGTP, 0.5 mM dCTP, 0.2 mM dTTP, 0.2 mM dUTP, 1 U Antarctic Thermolabile UDG, 500 ng Tte-UvrD Helicase, 150 ng Sso SSB, 150 ng Gst Polymerase, 20 U Roche RT, 800 ng RNAseHII, 20 U Endonuclease VIII, 0.1 U TseI. 80 nM eF2, 64 nM R3, 12 nM R12, 80 nM Probe7 ROX, 2 mM DTT, 2% Sucrose, 2.5% Dextran, 25 mg BSA, 0.14% Tween20 and 50 uL of lysed cells.

The Cfx program was 37° C. for 20 minutes followed by a 64° C. step for 80 minutes with ROX fluorescent reads every 30 seconds to monitor test probe cleavage.

Strong amplification of both serotypes tested was overserved at 10 4 and 10 5 CFU/reaction (FIG. 6).

### Example 6: L. monocytogenes Detection Assay with Mechanical Lysis

Mechanical bead lysis, an alternative lysis procedure to that of Example 5, was investigated in the present example. Zirconia Silicate beads with 0.1 and 0.5 mm diameter were purchased from Fisher Scientific. The Digital Disruptor Genie and the Digital Vortex Genie 2 with the TurboMix Attachment was used from Scientific Industries, Inc. Screw cap tubes (1.5 mL) used for bead-beating were purchased from USA Scientific. The optimal bead mass was determined in 1.5 mL screw-cap tubes with 200 μL of the serially diluted L. monocytogenes overnight culture in BPB to approximate 10⁷ CFU. 25 mg, 50 mg, 75 mg, and 100 mg all resulted in positive test signal with 50 mg producing the most desirable results with 2 minutes of bead beating. 50 uL of the lysate was added to lyophilized L. monocytogenes reactions.

To test mechanical lysis efficiencies, the following reagents were set up lyophilized pellets. 20 mM Tris pH 8.8, 10 mM MgSO₄, 10 mM KCL, 30 mM NaCl, 6.5 mM dATP, 0.5 mM dGTP, 0.5 mM dCTP, 0.2 mM dTTP, 0.2 mM dUTP, 1 U Antarctic Thermolabile UDG, 500 ng Tte-UvrD Helicase, 150 ng Sso SSB, 150 ng Gst Polymerase, 20 U Roche RT, 800 ng RNAseHII, 20 U Endonuclease VIII, 0.1 U TseI. 80 nM eF2, 64 nM R3, 12 nM R12, 80 nM Probe7 ROX, 2 mM DTT, 2% Sucrose, 2.5% Dextran, 25 mg BSA, 0.14% Tween20 and 50 μL of lysed cells.

The Cfx program was 37° C. for 20 minutes followed by a 64° C. step for 50 minutes with ROX fluorescent reads every 30 seconds to monitor test probe cleavage.

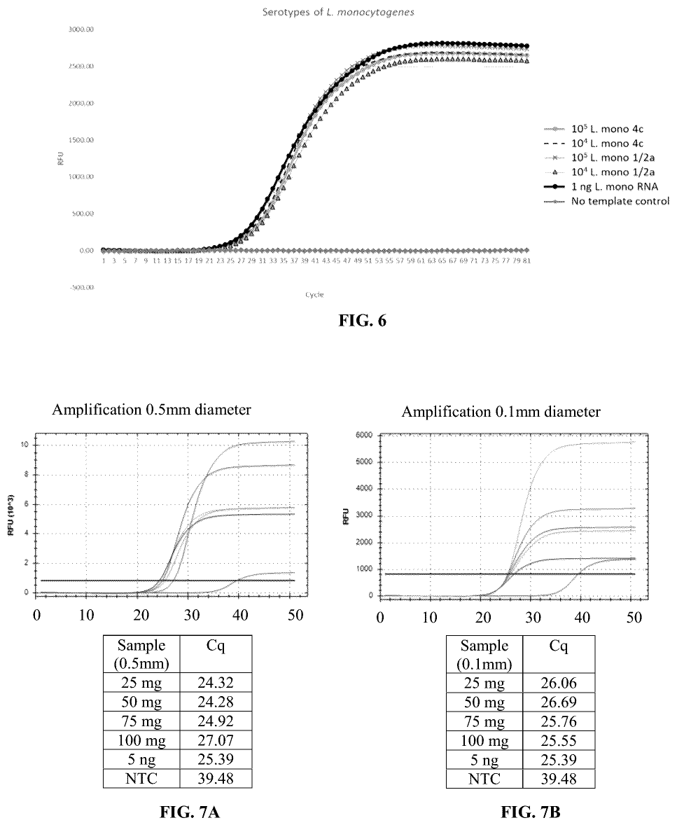

While both 0.1 mm and 0.5 mm diameter Zirconia Silicate beads showed similar efficiencies, the 0.5 mm diameter Zirconia Silicate beads produced amplification curves with greater max Relative Fluorescent Units (RFUs) than the 0.1 mm diameter Zirconia Silicate bead (FIGS. 7A and 7B).

Limit of detection with mechanical bead lysis and different disruption time was also tested. Disruption time was tested at 2 and 5 minutes. 200 uL of serially diluted L. monocytogenes overnight cultures were lysed with 50 mg Zirconia Silica beads for 2 minutes or 5 minutes. To confirm CFU levels, dilutions were plated on Typtic Soy Agar (TSA) plates and incubated at 37° C. overnight.

To test the limit of detection with mechanical bead lysis, the following reagents were set up lyophilized reactions. 20 mM Tris pH 8.8, 10 mM MgSO₄, 10 mM KCL, 30 mM NaCl, 6.5 mM dATP, 0.5 mM dGTP, 0.5 mM dCTP, 0.2 mM dTTP, 0.2 mM dUTP, 1 U Antarctic Thermolabile UDG, 500 ng Tte-UvrD Helicase, 150 ng Sso SSB, 150 ng Gst Polymerase, 20 U Roche RT, 800 ng RNAseHII, 20 U Endonuclease VIII, 0.1 U TseI. 80 nM eF2, 64 nM R3, 12 nM R12, 80 nM Probe7 ROX, 2 mM DTT, 2% Sucrose, 2.5% Dextran, 25 mg BSA, 0.14% Tween20 and 50 uL of lysed cells.

The Cfx program was 37° C. for 20 minutes followed by a 64° C. step for 120 minutes with ROX fluorescent reads every 30 seconds to monitor test probe cleavage.

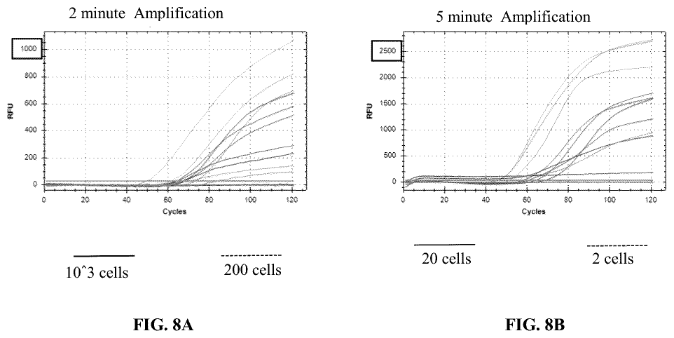

The 5-minute samples had slightly improved Cq over the 2-minute samples and greater max RFU (FIGS. 8A and 8B).

### Example 7: L. monocytogenes Detection Assay with Semi-Competitive Control

The present example demonstrates implementation of an L. monocytogenes detection assay with a modified RNA template as an internal positive control. Two types of modifications were used in the control template to prevent nuclease degradation: each base was fluorinated and five adenine bases with phosphorothioate linkages were placed at the 3′ and 5′ ends of the RNA template to further protect the RNA template. In Table 1B above, fluorinated bases are annotated as ‘f’ and phosphorothioate linkages as [Ps]. The control template is based on a semi-competitive design, utilizing the L. monocytogenes blocked forward primer, eF2, and a unique reverse primer SCR4 (Table 1B). The control probe and reverse primer were designed to ensure no unwanted interactions with the test probe.

To test the semi-competitive control of the assay, the following reagents were set up in lyophilized reactions. 20 mM Tris pH 8.8, 10 mM MgSO₄, 10 mM KCL, 30 mM NaCl, 6.5 mM dATP, 0.5 mM dGTP, 0.5 mM dCTP, 0.2 mM dTTP, 0.2 mM dUTP, 1 U Antarctic Thermolabile UDG, 500 ng Tte-UvrD Helicase, 150 ng Sso SSB, 150 ng Gst Polymerase, 20 U Roche RT, 800 ng RNAseHII, 20 U Endonuclease VIII, 0.1 U TseI. 80 nM eF2, 64 nM R3, 12 nM R12, 80 nM Probe7 ROX, 80 nM LM_CP5-HEX, 5,000 copies SC4, 2 mM DTT, 2% Sucrose, 2.5% Dextran, 25 mg BSA, 0.14% Tween20 and serially diluted L. monocytogenes RNA or L. innocua RNA.

The Optigene program was 37° C. for 20 minutes followed by a 64° C. step for 100 minutes with ROX and HEX fluorescent reads every 30 seconds to monitor test and control probe cleavage.

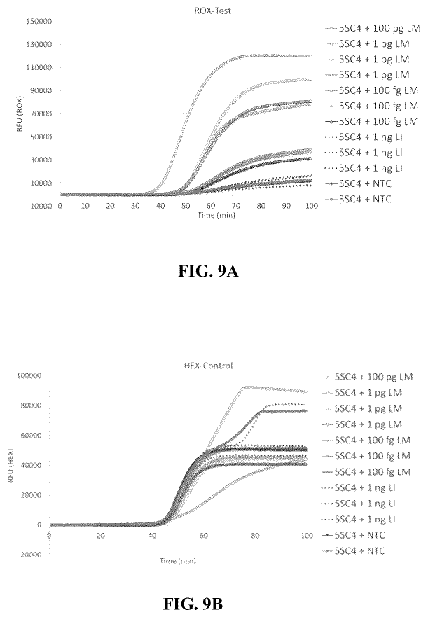

Addition of 5,000 copies of the semi-competitive control template per reaction allowed for detection of 100 fg of purified L. monocytogenes RNA while maintaining strong control signal in a reaction with a 1 ng of L. innocua RNA (FIGS. 9A and 9B).

As demonstrated by the present example, a modified RNA template can be used as an internal positive control for L. monocytogenes detection assays.

### Example 8: Sequences and Sequence Alignments

Additional sequences of the present disclosure are provided in Table 6. The blocking group of the blocked primers in Table 6 comprises rDXXDM, wherein R is an RNA residue, D is a DNA residue, M is a mismatched residue and x is a spacer. The mismatched residue (M) is underlined.

TABLE 6
Oligonucleotide sequences

| SEQ ID NO. | Oligo Name | Sequence 5′-3′ | Description |
|---|---|---|---|
| 1 (+2 Spacers, | eF2 | GGC TAC ACA CGT GCT ACA ATG GAT | Blocked Forward |
| each iSPC3, | | [rA]G/iSpC3//iSpC3/TG | Primer-rDXXDM |
| plus thymine | | | |
| and guanine) | | | |
| 2 (+2 Spacers, | R3 | GCA GCC TAC AAT CCG AAC TGA GAA T [rA]G/ | Blocked Reverse |
| each iSPC3, | | iSpC3//iSpc3/TC | Primer-rDXXDM |
| plus thymine | | | |
| and cytosine) | | | |
| 3 | R12 | CGA GTT GCA GCC TAC AAT CCG AAC TGA G | Unblocked Reverse |
| | | | Primer |
| 5 | Probe7 | /56-ROXN/CCT CGC GG[rC] TTC GC/31ABKFQ | Test Probe |
| (+fluorophore | ROX | | |
| and | | | |
| quencher) | | | |
| 6 | Hybrid- | GCGAAGCCGCGAGG | Probe hybridizes to |
| | ization | | this nucleic acid |
| | sequence | | sequence |
| 7 | LM_CP5- | /HEX/AGA GAG TCT A[rG]T ATC GAC ATG | Control Probe |
| (+fluorophore | HEX | GAT/BHQ-1 | |
| and | | | |
| quencher) | | | |
| 8 | SCR4 | CCGACTTCACCTGCTCGAGTAAAT | Control Reverse |
| | | | Primer |
| 9 | Lm_SC4 | [fA][Ps][fA][Ps][fA][Ps][fA][Ps][fA][Ps][fG][fG][fC] | Fluorinated RNA |
| | | [fU][fA][fC][fA][fC][fA][fC][fG][fU][fG][fC][fU] | control Template. |
| | | [fA][fC][fA][fA][fU][fG][fG][fA][fU][fA][fG][fU] | The annotation ″f″ |
| | | [fA][fC][fA][fA][fA][fA][fC][fA][fG][fA][fG][fA] | preceding a residue |
| | | [fG][fA][fG][fU][fC][fU][fA][fG][fU][fA][fU][fC] | in brackets denotes a |
| | | [fG][fA][fC][fA][fU][fG] | fluorinated residue. |
| | | [fG][fA][fU][fU][fA][fU][fU][fU][fA][fC][fU][fC][fG] | Phosphorothioate |
| | | [fA][fG][fC][fA][fG][fG][fU][fG][fA][fA][fG][fU][fC] | linkages are |
| | | [fG][fG][Ps][fA][Ps][fA][Ps][fA][Ps][fA][Ps] | annotated as [Ps]. |
| 12 | LM_GB_ | CGGTGCAAGCCGGAGGAAGGTGGGGATGACGTCAA | Partial L. |
| | 16S | ATCATCATGCCCCTTATGACCTGGGCTACACACGTGC | monocytogenes 16S |
| | | TACAATGGATAGTACAAAGGGTCGCGAAGCCGCGAG | sequence ordered |
| | | GTGGAGCTAATCCCATAAAACTATTCTCAGTTCGGAT | from IDT as a gene |
| | | TGTAGGCTGCAACTCGCCTACATGAAGCCGGAATCG | block fragment (250 |
| | | CTAGTAATCGTGGATCAGCATGCCACGGTGAATACG | bp) |
| | | TTCCCGGGCCTTGTACACACCGCCCGTCACACC | |
| 13 | LI_GB_ | ACTCAAAGGAATTGACGGGGGCCCGCACAAGCGGT | Partial L. innocua 16S |
| | 16S | GGAGCATGTGGTTTAATTCGAAGCAACGCGAAGAAC | sequence ordered |
| | | CTTACCAGGTCTTGACATCCTTTGACCACTCTGGAGA | from IDT as a gene |
| | | CAGAGCTTTCCCTTCGGGGACAAAGTGACAGGTGGT | block fragment (250 |
| | | GCATGGTTGTCGTCAGCTCGTGTCGTGAGATGTTGG | bp) |
| | | GTTAAGTCCCGCAACGAGCGCAACCCTTGATTTTAGT | |
| | | TGCCAGCATTTAGTTGGGCACTCTAAAGTGACT | |
| 14 | HDA_F1 | AGCCGAATTCAAAACATCGTAACTGAG | Forward primer as a |
| | | | positive control of |
| | | | HDA components |
| 15 | HDA_R1 | AATATTTTCCAACAACGCTTCTGCAAT | Reverse primer as a |
| | | | positive control of |
| | | | HDA components |
| 16 | HDA_GB | GTATCCTGAATCAGCCGAATTCAAAACATGGTAACTG | Gene block fragment |
| | | AGGATGATTTGATAACTTGGTACGAAAAAGCATTGA | for positive control |
| | | GAGGCCAATATCCGATTCGAGAAGCGTTGTTGGAAA | of |
| | | ATATTCTAGGTCATTCG | HDA components |

What is claimed is:

1-9. (canceled)

10. A composition comprising (i) an enzyme that binds uracil in a DNA strand and converts it into an apurinic site; (ii) an enzyme that cleaves DNA at apurinic sites; and (iii) a specialized dNTP that is recognized by the enzyme of (i).

11-53. (canceled)

54. A probe for detecting a nucleic acid of interest, said probe comprising the nucleic acid sequence CCT CGC GG[rC] TTC GC (SEQ ID NO: 5), or a sequence with at least 90% sequence identity thereto, wherein the probe comprises DNA bases and a single RNA base, and wherein the “r” denotes that the base is RNA.

55. The probe of claim 54, wherein the probe is a fluorescent molecular probe.

56. The probe of claim 54, wherein the probe comprises a fluorophore and a quencher, and wherein the quencher prevents the fluorophore from generating fluorescence unless the probe is bound to a nucleic acid molecule of interest.

57. The probe of claim 56, wherein the probe comprises a linked fluorophore at the 5′ end of the probe and linked quencher at the 3′ end of the probe.

58. A conditional fluorescent hybridization probe that emits fluorescence when hybridized to a nucleic acid molecule comprising the nucleic acid sequence of: GCG AAG CCG CGA GG (SEQ ID NO: 6).

59. A composition comprising the probe of claim 54 or 58.

60. The composition of claim 59, further comprising:
(i) a primer for initiating reverse transcription of an RNA target and polymerization of cDNA; and/or
(ii) one or more primers for target nucleic acid amplification.

61. A primer comprising:
(i) an oligonucleotide comprising the sequence GGC TAC ACA CGT GCT ACA ATG GAT (SEQ ID NO: 10); and
(ii) a blocking group that prevents primer extension and/or inhibits the blocked primer from serving as a template for DNA synthesis.

62. A primer comprising:
(i) an oligonucleotide comprising the sequence GCA GCC TAC AAT CCG AAC TGA GAA T (SEQ ID NO: 11); and
(ii) a blocking group that prevents primer extension and/or inhibits the blocked primer from serving as a template for DNA synthesis.

63. The primer of claim 61 or 62, wherein the blocking group is linked at or near the 3′ end of the oligonucleotide.

64. The primer of claim 61 or 62, wherein the blocking group comprises rDXXDM, wherein r is a ribobase, D is a DNA base, M is a mismatched residue to a target nucleic acid sequence, and x is a C3 spacer.

65. The primer of claim 64, wherein, wherein
(i) the ribobase is an adenine base;
(ii) the DNA base is a guanine base or a thymine base;
(iii) there are two C3 spacers; and
(iv) the mismatched residue is a guanine or cytosine base.

66. A composition comprising a primer selected from the group consisting of the primer of claim 61 and the primer of claim 62.

67. The composition of claim 66, comprising the primer of claim 61 and the primer of claim 62.

68. The composition of claim 66, further comprising an-oligonucleotide primer having the sequence: CGA GTT GCA GCC TAC AAT CCG AAC TGA G (SEQ ID NO: 3).

69. The composition of 68, further comprising the probe of claim 54 or 58.

70. A kit comprising a primer selected from the group consisting of the primer of claim 61 and the primer of claim 62.

71. The kit of claim 70, further comprising an oligonucleotide primer having the sequence: CGA GTT GCA GCC TAC AAT CCG AAC TGA G (SEQ ID NO: 3).

72. The kit of claim 71, further comprising the probe of claim 54 or 58.

73. A probe for detecting a nucleic acid of interest, said probe comprising the nucleic acid sequence of: AGA GAG TCT A[rG]T ATC GAC ATG GAT (SEQ ID NO: 7), or a sequence with at least 90% sequence identity thereto, wherein the probe comprises DNA bases and a single RNA base, and wherein the “r” preceding a residue in brackets denotes that the base is RNA.

74. An RNA oligonucleotide comprising or consisting of the sequence of: [fA][Ps][fA][Ps][fA][Ps][fA][Ps][fA][Ps][fG][fG][fC] [fU][fA][fC] [fA][fC][fA] [fC][fG][fU] [fG][fC][fU] [fA][fC][fA] [fA][fU][fG] [fG][fA][fU] [fA][fG][fU] [fA][fC][fA] [fA][fA][fA] [fC][fA][fG] [fA][fG][fA] [fG][fA][fG] [fU][fC][fU] [fA][fG][fU] [fA][fU][fC] [fG][fA][fC] [fA][fU][fG] [fG][fA][fU] [fU][fA][fU] [fU][fU][fA] [fC][fU][fC] [fG][fA][fG] [fC][fA][fG] [fG][fU][fG] [fA] [fA] [fG] [fU][fC][fG] [fG] [Ps] [fA] [Ps] [fA] [Ps] [fA] [Ps] [fA] [Ps] (SEQ ID NO: 9), wherein “f” preceding a residue in brackets denotes a fluorinated residue and 5′ and 3′ adenine bases have phosphorothioate linkages [Ps].


SEQUENCE LISTING (40 sequences)

SEQ ID NO: 1 - DNA, 26 bp, synthetic construct
  misc_feature 25: note = RNA ribobase
  source 1..26: mol_type = other DNA
ggctacacacgtgctacaatggatag

SEQ ID NO: 2 - DNA, 27 bp, synthetic construct
  misc_feature 26: note = RNA ribobase
  source 1..27: mol_type = other DNA
gcagcctacaatccgaactgagaatag

SEQ ID NO: 3 - DNA, 28 bp, synthetic construct
  source 1..28: mol_type = other DNA
cgagttgcagcctacaatccgaactgag

SEQ ID NO: 4 - DNA, 94 bp, synthetic construct
  source 1..94: mol_type = other DNA
ggctacacacgtgctacaatggatagtacaaagggtcgcgaagccgcgaggtggagctaatcccataaaactattctcagttcggattgtaggc

SEQ ID NO: 5 - DNA, 14 bp, synthetic construct
  misc_feature 9: note = RNA ribobase
  source 1..14: mol_type = other DNA
cctcgcggcttcgc

SEQ ID NO: 6 - DNA, 14 bp, synthetic construct
  source 1..14: mol_type = other DNA
gcgaagccgcgagg

SEQ ID NO: 7 - DNA, 24 bp, synthetic construct
  misc_feature 11: note = RNA
  source 1..24: mol_type = other DNA
agagagtctagtatcgacatggat

SEQ ID NO: 8 - DNA, 24 bp, synthetic construct
  source 1..24: mol_type = other DNA
ccgacttcacctgctcgagtaaat

SEQ ID NO: 9 - RNA, 94 nt, synthetic construct
  source 1..94: mol_type = other RNA
  misc_feature 1..>94: note = fluorinated
  misc_feature 1..>6: note = phosphorothioate linkage
  misc_feature 90..>94: note = phosphorothioate linkage
aaaaaggctacacacgtgctacaatggatagtacaaaacagagagagtctagtatcgacatggattatttactcgagcaggtgaagtcggaaaa

SEQ ID NO: 10 - DNA, 24 bp, synthetic construct
  source 1..24: mol_type = other DNA
ggctacacacgtgctacaatggat

SEQ ID NO: 11 - DNA, 25 bp, synthetic construct
  source 1..25: mol_type = other DNA
gcagcctacaatccgaactgagaat

SEQ ID NO: 12 - RNA, 250 nt, synthetic construct
  source 1..250: mol_type = other RNA
cggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgaatacgttcccgggccttgtacacaccgcccgtcacacc

SEQ ID NO: 13 - RNA, 250 nt, synthetic construct
  source 1..250: mol_type = other RNA
actcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgact

SEQ ID NO: 14 - DNA, 27 bp, synthetic construct
  source 1..27: mol_type = other DNA
agccgaattcaaaacatcgtaactgag

SEQ ID NO: 15 - DNA, 27 bp, synthetic construct
  source 1..27: mol_type = other DNA
aatattttccaacaacgcttctgcaat

SEQ ID NO: 16 - DNA, 126 bp, synthetic construct
  source 1..126: mol_type = other DNA
gtatcctgaatcagccgaattcaaaacatggtaactgaggatgatttgataacttggtacgaaaaagcattgagaggccaatatccgattcgagaagcgttgttggaaaatattctaggtcattcg

SEQ ID NO: 17 - RNA, 1394 nt, L. monocytogenes
  source 1..1394: mol_type = rRNA
gaacgaacggaggaagagcttgctcttccaaagttagtggcggacgggtgagtaacacgtgggcaacctgcctgtaagttggggataactccgggaaaccggggctaataccgaataataaagwgtggcgcatgccacgcttttgaaagatggtttcggctatcacttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgaatacgttcccgggccttgtacacaccgcccgtcacaccacgagagtttgtaacacccgaagtcggtagggtaacct

SEQ ID NO: 18 - RNA, 1394 nt, L. marthii serotype 1
  source 1..1394: mol_type = rRNA
gaacgaacggaggaagagcttgctcttccaaagttagtggcggacgggtgagtaacacgtgggcaacctgcctgtaagttggggataactccgggaaaccggggctaataccgaataataagatgtggcgcatgccacgcctttgaaagatggtttcggctatcacttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttaattttagttgccagcatttagttgggcactctaaagtgactgccggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgaatacgttcccgggccttgtacacaccgcccgtcacaccacgagagtttgtaacacccgaagtcggtagggtaacct

SEQ ID NO: 19 - RNA, 1394 nt, L. marthii serotype 2
  source 1..1394: mol_type = rRNA
gaacgaacggaggaagagcttgctcttccaaagttagtggcggacgggtgagtaacacgtgggcaacctgcctgtaagttggggataactccgggaaaccggggctaataccgaataataagatgtggcgcatgccacgcctttgaaagatggtttcggctatcacttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggakggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttaattttagttgccagcatttagttgggcactctaaagtgactgccggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgaatacgttcccgggccttgtacacaccgcccgtcacaccacgagagtttgtaacacccgaagtcggtagggtaacct

SEQ ID NO: 20 - RNA, 1394 nt, L. marthii serotype 3
  source 1..1394: mol_type = rRNA
gaacgaacggaggaagagcttgctcttccaawgttagtggcggacgggtgagtaacacgtgggcaacctgcctgtaagttggggataactccgggaaaccggggctaataccgaataataagatgtggcgcatgccacgcctttgaaagatggtttcggctatcacttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttaattttagttgccagcatttagttgggcactctaaagtgactgccggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgaatacgttcccgggccttgtacacaccgcccgtcacaccacgagagtttgtaacacccgaagtcggtagggtaacct

SEQ ID NO: 21 - RNA, 1394 nt, L. Marthii serotype 4
  source 1..1394: mol_type = rRNA
gaacgaacggaggaagagcttgctcttccaaagttagtggcggacgggtgagtaacacgtgggcaacctgcctgtaagttggggataactccgggaaaccggggctaataccgaataataagatgtggcgcatgccacgcctttgaaagatggtttcggctatcacttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttaattttagttgccagcatttagttgggcactctaaagtgactgccggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgaatacgttcccgggccttgkacacaccgsccgtcacaccacgagagtttgtaacacccgaagtcggtagggtaacct

SEQ ID NO: 22 - RNA, 1394 nt, L. mono 1/2a 3 (1/6)
  source 1..1394: mol_type = rRNA
gaacgaacggaggaagagcttgctcttccaatgttagtggcggacgggtgagtaacacgtgggcaacctgcctgtaagttggggataactccgggaaaccggggctaataccgaatgataaagtgtggcgcatgccacgcttttgaaagatggtttcggctatcgcttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgaatacgttcccgggccttgtacacaccgcccgtcacaccacgagagtttgtaacacccgaagtcggtagggtaacct

SEQ ID NO: 23 - RNA, 1394 nt, L. mono 1a (wgs)
  source 1..1394: mol_type = rRNA
gaacgaacggaggaagagcttgctcttccaaagttagtggcggacgggtgagtaacacgtgggcaacctgcctgtaagttggggataactccgggaaaccggggctaataccgaatgatagaatgtggcgcatgccacgcttttgaaagatggtttcggctatcgcttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgaatacgttcccgggccttgtacacaccgcccgtcacaccacgagagtttgtaacacccgaagtcggtagggtaacct

SEQ ID NO: 24 - RNA, 1394 nt, L. mono 3a 2 (2/6)
  source 1..1394: mol_type = rRNA
gaacgaacggaggaagagcttgctcttccaaagttagtggcggacgggtgagtaacacgtgggcaacctgcctgtaagttggggataactccgggaaaccggggctaataccgaatgatagagtgtggcgcatgccacgcttttgaaagatggtttcggctatcgcttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgaatacgttcccgggccttgtacacaccgcccgtcacaccacgagagtttgtaacacccgaagtcggtagggtaacct

SEQ ID NO: 25 - RNA, 1394 nt, L. mono 3a 5 (1/6)
  source 1..1394: mol_type = rRNA
gaacgaacggaggaagagcttgctcttccaaagttagtggcggacgggtgagtaacacgtgggcaacctgcctgtaagttggggataactccgggaaaccggggctaataccgaatgatagagtgtggcgcatgccacgcttttgaaagatggtttcggctatcgcttacagatgggcccgcggtgcattagctagttggtagggtaatggcctacccaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcctggatcagcatgccacggtgaatacgttcccgggccttgtacacaccggccgtcacaccacgagagtttgtaacacccgaagtcggtagggtaacct

SEQ ID NO: 26 - RNA, 1394 nt, L. mono 3b (6/6)
  source 1..1394: mol_type = rRNA
gaacgaacggaggaagagcttgctcttccaaagttagtggcggacgggtgagtaacacgtgggcaacctgcctgtaagttggggataactccgggaaaccggggctaataccgaatgataaagtgtggcgcatgccacgcttttgaaagatggtttcggctatcgcttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatagtacaaagggtcgcgaagccgcgaggtggagctaatcccataaaactattctcagttcggattgtaggctgcaactcgcctacatgaagccggaatcgctagtaatcgtggatcagcatgccacggtgaatacgttcccgggccttgtacacaccgcccgtcacaccacgagagtttgtaacacccgaagtcggtagggtaacct

SEQ ID NO: 27 - RNA, 1394 nt, L. innocua 6a
  source 1..1394: mol_type = rRNA
gaacgaacggaggaagagcttgctcttccaaagttagtggcggacgggtgagtaacacgtgggcaacctgcctgtaagttggggataactccgggaaaccggggctaataccgaatgatagagtgtggcgcatgccacgctcttgaaagatggtttcggctatcgcttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatggtacaaagggtcgcgaagccgcgaggtggagccaatcccataaaaccattctcagttcggattgtaggctgcaactcgcctacatgaagccggaatcgctagtaatcgtggatcagcatgccacggtgaatacgttcccgggccttgtacacaccgcccgtcacaccacgagagtttgtaacacccgaagtcggtagggtaacct

SEQ ID NO: 28 - RNA, 1394 nt, L. innocua 6b
  source 1..1394: mol_type = rRNA
gaacgaacggaggaagagcttgctcttccaatgttagtggcggacgggtgagtaacacgtgggcaacctgcccgtaagttggggataactccgggaaaccggggctaataccgaataatagagtgtggcgcatgccacactcttgaaagatggtttcggctatcgcttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccccgtgtatcgctgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttaattttagttgccagcatttagttgggcactctaaagtgactgccggtgcaagccggaggaaggtggggatgacgtcaaatcatcatgccccttatgacctgggctacacacgtgctacaatggatggtacaaagggtcgcgaagccgcgaggtggagccaatcccataaaaccattctcagttcggattgtaggctgcaactcgcctacatgaagccggaatcgctagtaatcgtggatcagcatgccacggtgaatacgttcccgggccttgtacacaccgcccgtcacaccacgagagtttgtaacacccgaagtcggtagggtaacct

SEQ ID NO: 29 - RNA, 1394 nt, L. seeligeri 1/2b 1/6
  source 1..1394: mol_type = rRNA
gaacgaacggaggaagagcttgctcttccaaagttagtggcggacgggtgagtaacacgtgggcaacctgcctgtaagttggggataactccgggaaaccggggctaataccgaatgataaggagtgacgcatgtcactgctttgaaagatggtttcggctatcgcttatagatgggcccgcggtgcattagctagttggtagggtaaaggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatggtacaaagggtagcgaagccgcgaggtggagccaatcccataaaaccattctcagttcggattgtaggctgcaactcgcctacatgaagctggaatcgctagtaatcgtggatcagcatgccacggtgaatacgttcccgggccttgtacacaccgcccgtcacaccacgagagtttgtaacacccgaagtcggtagggtaacct

SEQ ID NO: 30 - RNA, 1394 nt, L. ivanovii 5
  source 1..1394: mol_type = rRNA
gaacgaacggaggaagagcttgctcttccaatgttagtggcggacgggtgagtaacacgtgggcaacctgcctgtaagttggggataactccgggaaaccggggctaataccgaatgataagtagtgacgcatgtcatcactttgaaagatggtttcggctatcgcctacagatgggcccgcggtgcattagctagttggtagggtaaaggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtatgaagaaggttttcggatcgtaaagtactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgcgcgcaggcggtcttttaagtctgatgtgaaagcccccggcttaaccggggagggtcattggaaactggaagactggagtgcagaagaggagagtggaattccacgtgtagcggtgaaatgcgtagatatgtggaggaacaccagtggcgaaagcgactctctgttctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcgaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatggtacaaagggtcgcgaagccgcgaggtggagccaatcccataaaaccattctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtagggtaacct

SEQ ID NO: 31 - RNA, 1361 nt, L. floridensis
  source 1..1361: mol_type = rRNA
gtgagtggcggacgggtgagtaacacgtgggcaacctgcctatcagcgggggataacacttggaaacaggtgctaataccgcataacagtaaggttcgcatgaaccttgtttgaaagacggtttcggctgtcactgatagatgggcccgcggtgcattagctagttggtggggtaaaggcctaccaaggcgacgatgcatagccgacctgagagggtgatcggccacactgggactgagacacggcccagactcctacgggaggcagcagtagggaatcttccgcaatggacgaaagtctgacggagcaacgccgcgtgtgtgaagaaggttttcggatcgtaaagcactgttgttagagaagaacaaggagaaggtaactacttctcccctgacggtatctaaccagaaagccacggctaactacgtgccagcagccgcggtaatacgtaggtggcaagcgttgtccggaattattgggcgtaaagcgcgcgcaggcggtttcttaagtctgatgtgaaagcccccggcttaaccggggagggtcattggaaactgggagactggagtgcagaagaggagagtggaattccatgtgtagcggtgaaatgcgtagatatatggaggaacaccagtggcgaaggcggctctctggtctgtaactgacgctgaggcgcgaaagcgtggggagcaaacaggattagataccctggtagtccacgccgtaaacgatgagtgctaagtgttagggggtttccgccccttagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagcttccccttcgggggcaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatggtacaaagggtagcgaagccgcgaggtggagccaatcccataaaaccattctcagttcggattgcaggctgcaactcgcctgcatgaagccggaatcgctagtaatcgcggatcagcatgccgcggtgaatacgttcccgggccttgtacacaccgcccgtcacaccacgagagtttgtaacacccgaagtcggtagggtaacct

SEQ ID NO: 32 - RNA, 1360 nt, L. fleischmannii
  source 1..1360: mol_type = rRNA
gtgagtggcggacgggtgagtaacacgtgggcaacctgcctatcagcgggggataacacttggaaacaggtgctaataccgcataatagagaagttcgcatgaacttcttttgaaagacggtttcggctgtcactgatagatgggcccgcggtgcattagctagttggtggggtaaaggcctaccaaggcgacgatgcatagccgacctgagagggtgatcggccacactgggactgagacacggcccagactcctacgggaggcagcagtagggaatcttccgcaatggacgaaagtctgacggagcaacgccgcgtgtgtgaagaaggttttcggatcgtaaagcactgttgttagagaagaacaaggataagagtcactgcttgtcccttgacggtatctaaccagaaagccacggctaactacgtgccagcagccgcggtaatacgtaggtggcaagcgttgtccggaattattgggcgtaaagcgcgcgcaggcggtttcttaagttgggtgtgaaagccctcggctcaaccgaggagggtcactcaaaactgggagactggagtgcagaagaggagagtggaattccatgtgtagcggtgaaatgcgtagatatatggaggaacaccagtggcgaaggcggctctctggtctgtaactgacgctgaggcgcgaaagcgtggggagcaaacaggattagataccctggtagtccacgccgtaaacgatgagtgctaagtgttagggggtttccgccccttagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagcttccccttcgggggcaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatggtacaaagggcagcgaagccgtgaggtgaagccaatcccataaaaccattctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtagggtaac

SEQ ID NO: 33 - RNA, 1361 nt, L. aquatica
  source 1..1361: mol_type = rRNA
gtgagtggcggacgggtgagtaacacgtgggcaacctgcctatcagcgggggataacacttggaaacaggtgctaataccgcataacagagaggttcgcatgaaccttttttgaaagacggtttcggctgtcactgatagatgggcccgcggtgcattagctagttggtggggtaaaggcctaccaaggccacgatgcatagccgacctgagagggtgatcggccacactgggactgagacacggcccagactcctacgggaggcagcagtagggaatcttccgcaatggacgaaagtctgacggagcaacgccgcgtgtgtgaagaaggttttcggatcgtaaagcactgttgttagagaagaacaaggatgagttaactattcatcccctgacggtatctaaccagaaagccacggctaactacgtgccagcagccgcggtaatacgtaggtggcaagcgttgtccggaattattgggcgtaaagcgcgcgcaggcggtttcttaagttgggtgtgaaagccctcggctcaaccgaggagggtcactcaaaactgggagactggagtgcagaagaggagagtggaattccatgtgtagcggtgaaatgcgtagatatatggaggaacaccagtggcgaaggcggctctctggtctgtaactgacgctgaggcgcgaaagcgtggggagcaaacaggattagataccctggtagtccacgccgtaaacgatgagtgctaagtgttagggggtttccgccccttagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagcttccccttcgggggcaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatggaacaaagggyagcgaagccgcgaggtgaagccaatcccataaaaccattctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtagggtaacct

SEQ ID NO: 34 - RNA, 1363 nt, L. grayi
  source 1..1363: mol_type = rRNA
gttagtggcggacgggtgagtaacacgtgggcaacctgcctgtaagattgggataactccgggaaaccggagctaataccgaataataatcagctccgcatggagcaggtttgaaaggcggcttcggctgtcacttacagatgggcccgcggtgcattagctagttggtggggtaaaggcctaccaaggcgacgatgcatagccgacctgagagggtgatcggccacactgggactgagacacggcccagactcctacgggaggcagcagtagggaatcttccgcaatggacgaaagtctgacggagcaacgccgcgtgtgtgaagaaggttttcggatcgtaaagcactgttgttagagaagaacaaggataagagtaactgcttgtcccttgacggtatctaaccagaaagccacggctaactacgtgccagcagccgcggtaatacgtaggtggcaagcgttgtccggaattattgggcgtaaagcgcgcgcaggcggtttcttaagtctgatgtgaaagcccccggcttaaccggggagggtcattggaaactgggagacttagagtgcagaagaggagagtggaattccatgtgtagcggtgaaatgcgtagatatatggaggaacaccagtggcgaaggcgactctctggtctgtaactgacgctgaggcgcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggatgatacaaagggtcgcgaagccgcgaggtgaagctaatcccataaaatcattctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtagggtaacct

SEQ ID NO: 35 - RNA, 1396 nt, L. rocourtiae
  source 1..1396: mol_type = rRNA
gaacgaaccgacgaaagagcttgctctttctgacgttagtggcggacgggtgagtaacacgtgggcaacctgcctgtaagttggggataactccgggaaaccggggctaataccgaataataacaagcgtcgcatgacgcctgtttgaaagacggtttcggctgtcacttacagatgggcccgcggtgcattagctagttggtagggtaatggcctaccaaggcgacgatgcatagccgacctgagagggtgatcggccacactgggactgagacacggcccagactcctacgggaggcagcagtagggaatcttccgcaatggacgaaagtctgacggagcaacgccgcgtgtgtgaagaaggttttcgaatcgtaaagcactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggcttaaccggggagggtcattggaaactgagagacttgaatgcaggagaggtaagtggaattccacgtgtagcggtgaaatgcgtagatatgtggaggaacaccagtggcgaaggcggcttactggcctgtaattgacgctgaggctcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagtggtggagcatgtggtttaattcgaagcaacgcgaagaaccttaccaggtcttgacatcctttgaaccttctggagacagaactttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggataatacaaagggttgccaaaccgcgaggtcgagctaatcccataaaattattctcagttcggattgtaggctgcaactcgcctacatgaagccggaatcactagtaatcgtggatcagcatgccacggtgaatacgttcccgggccttgtacacaccgcccgtcacaccacgagagtttgtaacacccgaagtcggtagggtaacct

SEQ ID NO: 36 - RNA, 1381 nt, L. riparia
  source 1..1381: mol_type = rRNA
aaaagcttgcttttctgacgtgagtggcggacgggtgagtaacacgtgggcaacctgcctatcagttggggataactccgggaaaccggggctaataccgaataataacgagcgtcgcatgacgctggtttgaaagatggtttcggctatcgctgatagatgggcccgcggtgcattagctagttggtggggtaatggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgtgtgaagaaggttttcggatcgtaaagcactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggcttaaccggggagggtcattggaaactgagagacttgaatgcaggagaggtaagtggaattccacgtgtagcggtgaaatgcgtagatatgtggaggaacaccagtggcgaaggcggcttactggcctgtaattgacgctgaggctcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagtggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggataatacaaagggttgccaaaccgcgaggtcgagctaatcccataaaattattctcagttcggattgtaggctgcaactcgcctacatgaagccggaatcactagtaatcgtggatcagcatgccacggtgaatacgttcccgggccttgtacacaccgcccgtcacaccacgagagtttgtaacacccgaagtcggtagggtaacct

SEQ ID NO: 37 - RNA, 1396 nt, L. newyorkensis
  source 1..1396: mol_type = rRNA
gaacgaaccgacgaaagagcttgctctttctgacgttagtggcggacgggtgagtaacacgtgggcaacctgcctgtaagttggggataactccgggaaaccggggctaataccgaataataacaagcgtcgcatgacgcctgtttgaaagatggtttcggctatcacttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtgtgaagaaggttttcggatcgtaaagcactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggcttaaccggggagggtcattggaaactgagagacttgaatgcaggagaggtaagtggaattccacgtgtagcggtgaaatgcgtagatatgtggaggaacaccagtggcgaaggcggcttactggcctgtaattgacgctgaggctcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagtggtggagcatgtggtttaattcgaagcaacgcgaagaaccttaccaggtcttgacatcctttgaaccttctggagacagaactttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggataatacaaagggttgccaaaccgcgaggtcgagctaatcccataaaattattctcagttcggattgtaggctgcaactcgcctacatgaagccggaatcactagtaatcgtggatcagcatgccacggtgaatacgttcccgggccttgtacacaccgcccgtcacaccacgagagtttgtaacacccgaagtcggtagggtaacct

SEQ ID NO: 38 - RNA, 1396 nt, L. grandensis
  source 1..1396: mol_type = rRNA
gaacgaaccgaagaaagagcttgctctttcggacgttagtggcggacgggtgagtaacacgtgggcaacctgcctgtaagttggggataactccgggaaaccggggctaataccgaataataacaagcgtcgcatgacgcctgtttgaaagatggtttcggctatcacttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtgtgaagaaggttttcggatcgtaaagcactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggcttaaccggggagggtcattggaaactgagagacttgaatgcaggagaggtaagtggaattccacgtgtagcggtgaaatgcgtagatatgtggaggaacaccagtggcgaaggcggcttactggcctgtaattgacgctgaggctcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagtggtggagcatgtggtttaattcgaagcaacgcgaagaaccttaccaggtcttgacatcctttgaccattctggagacagaactttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggataatacaaagggttgccaaaccgcgaggtcgagctaatcccataaaattattctcagttcggattgtaggctgcaactcgcctacatgaagccggaatcactagtaatcgtggatcagcatgccacggtgaatacgttcccgggccttgtacacaccgcccgtcacaccacgagagtttgtaacacccgaagtcggtagggtaacct

SEQ ID NO: 39 - RNA, 1361 nt, L. cornellensis 1
  source 1..1361: mol_type = rRNA
gttagtggcggacgggtgagtaacacgtgggcaacctgcctgtaagttggggataactccgggaaaccggggctaataccgaataataacaagcgtcgcatgacgcctgtttgaaagatggtttcggctatcacttacagatgggcccgcggtgcattagctagttggtagggtaatggcctaccaaggcaacgatgcatagccgacctgagagggtgatcggccacactgggactgagacacggcccagactcctacgggaggcagcagtagggaatcttccgcaatggacgaaagtctgacggagcaacgccgcgtgtgtgaagaaggttttcggatcgtaaagcactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggcttaaccggggagggtcattggaaactgagagacttgaatgcaggagaggtaagtggaattccacgtgtagcggtgaaatgcgtagatatgtggaggaacaccagtggcgaaggcggcttactggcctgtaattgacgctgaggctcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagtggtggagcatgtggtttaattcgaagcaacgcgaagaaccttaccaggtcttgacatcctttgaccattctggagacagaactttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggataatacaaagggttgccaaaccgcgaggtcgagctaatcccataaaattattctcagttcggattgtaggctgcaactcgcctacatgaagccggaatcactagtaatcgtggatcagcatgccacggtgaatacgttcccgggccttgtacacaccgcccgtcacaccacgagagtttgtaacacccgaagtcggtagggtaacc

SEQ ID NO: 40 - RNA, 1394 nt, L. booriae
  source 1..1394: mol_type = rRNA
gaacgaaccgacgaaaagcttgcttttctgacgtgagtggcggacgggtgagtaacacgtgggcaacctgcctatcagttggggataactccgggaaaccggggctaataccgaataataacgggcgtcgcatgacgctggtttgaaagatggtttcggctatcgctgatagatgggcccgcggtgcattagctagttggtggggtaatggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgtgtgaagaaggttttcggatcgtaaagcactgttgttagagaagaacaaggataagagtaactgcttgtcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggcttaaccggggagggtcattggaaactgagagacttgaatgcaggagaggtaagtggaattccacgtgtagcggtgaaatgcgtagatatgtggaggaacaccagtggcgaaggcggcttactggcctgtaattgacgctgaggctcgaaagcgtggggagcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacgggggcccgcacaagtggtggagcatgtggtttaattcgaagcaacgcgaagaaccttaccaggtcttgacatcctttgaccactctggagacagagctttcccttcggggacaaagtgacaggtggtgcatggttgtcgtcagctcgtgtcgtgagatgttgggttaagtcccgcaacgagcgcaacccttgattttagttgccagcatttagttgggcactctaaagtgactgccggtgcaagccggaggaaggtggggatgacgtcaaatcatcatgccccttatgacctgggctacacacgtgctacaatggataatacaaagggttgccaaaccgcgaggtcgagctaatcccataaaattattctcagttcggattgtaggctgcaactcgcctacatgaagccggaatcactagtaatcgtggatcagcatgccacggtgaatacgttcccgggccttgtacacaccgcccgtcacaccacgagagtttgtaacacccgaagtcggtagggtaacct